(19) United States
(12) Patent Application Publication
(10) Pub. No.: US 2026/0136071 A1
(43) Pub. Date: May 14, 2026

(54) METHODS, DEVICES, APPARATUSES AND SYSTEMS FOR PROVIDING CONTENT TO VIEWERS FOR ENHANCING HEALTH CONDITIONS OF THE VIEWERS

(71) Applicants: Tarun Sunder Raj, Palawan, CA (US); Benjamin Tucker, Yakima, WA (US)

(72) Inventors: Tarun Sunder Raj, Palawan, CA (US); Benjamin Tucker, Yakima, WA (US)

(21) Appl. No.: 19/430,216

(22) Filed: Dec. 22, 2025

Related U.S. Application Data
(63) Continuation-in-part of application No. 17/843,647, filed on Jun. 17, 2022, now Pat. No. 12,506,932.
Application No. 17/843,647 is a continuation-in-part of application No. 17/196,536, filed on Mar. 9, 2021, now Pat. No. 11,368,235.
Application No. 17/196,536 is a continuation-in-part of application No. 15/654,581, filed on Jul. 19, 2017, now Pat. No. 10,945,014.
(60) Provisional application No. 62/364,146, filed on Jul. 19, 2016.

(51) Int. Cl. H04N 21/478; A61M 21/00; A61M 21/02; H04L 9/00; H04N 21/25; H04N 21/442

(52) U.S. Cl. CPC H04N 21/478; A61M 21/02; H04L 9/50; H04N 21/252; H04N 21/44218; A61M 2021/0022; A61M 2021/0027; A61M 2021/0044; A61M 2021/005; A61M 2021/0088

(57) ABSTRACT

The present disclosure provides a method for providing content to viewers for enhancing health conditions of the viewers. Further, the method includes receiving a media content, receiving a broadcaster preference, generating an augmented media content for the enhancing of the health conditions, and transmitting the augmented media content to a viewer device. Further, the augmented media content further includes two or more parameters of two or more augmented media contents. Further, the two or more parameters is configured to drive two or more sensory stimuli in the viewer. Further, the viewer device includes an audio output device configured for generating a sound wave based on the augmented media content, a visual output device configured for generating a light based on the augmented media content, a vibrotactile device configured for generating a vibration based on the augmented media content to drive the two or more sensory stimuli in the viewer.

### FIELD OF DISCLOSURE

Generally, the present disclosure relates to the field of data processing. More specifically, the present disclosure relates to methods, systems, apparatuses, and devices for providing content to viewers for enhancing health conditions of the viewers.

### BACKGROUND

Existing techniques for providing content to viewers for enhancing wellbeing of the viewers are deficient in regard to several aspects. For instance, current technologies do not consider viewers' responses during presenting the content. Furthermore, current technologies do not consider viewers' biometrics during presenting the content. Moreover, current technologies do not use viewers' health profiles for generating content.

The present disclosure generally relates to the field of adaptive media technologies, immersive content delivery environments, and dynamic multimodal experience-generation systems that interact with user-specific contextual information. The field is of significant importance because modern users increasingly rely on digital content not only for entertainment, but also for wellness, personalized therapeutic support, environmental awareness, contextual interaction, and enhanced engagement across virtual, augmented, and mixed-reality environments. As digital ecosystems continue to expand across mobile devices, wearable devices, haptic interfaces, extended-reality platforms, and distributed computing infrastructures, there is a growing demand for content delivery frameworks capable of responding intelligently to real-time conditions, user state changes, and multi-channel interaction patterns.

It is often desirable in the field to achieve an objective of delivering content experiences that adapt meaningfully to a user's moment-to-moment context and environment. Achieving the objective may allow for improved personalization, deeper engagement, enhanced therapeutic value, and more effective integration of digital content within a user's daily activities and wellbeing routines. However, existing systems that attempt to deliver or modify content often face significant challenges in achieving the objective. For example, many known platforms are limited in the ability to incorporate real-time contextual information from multiple sensors or sources, particularly when such information reflects rapid fluctuations in user state, environmental changes, or multi-modal interaction patterns. Certain conventional content-delivery systems may further struggle to coordinate complex combinations of signals across heterogeneous devices, thereby limiting the capacity to generate immersive, synchronized, or therapeutically relevant experiences. Additional difficulties may arise when attempting to integrate adaptive logic, machine-driven personalization, or dynamically sourced augmentation elements into content workflows that must operate consistently across distributed networks, diverse hardware configurations, or latency-sensitive environments.

Other limitations may be observed in frameworks that attempt to incorporate user-generated data, wellness-related information, or extended-reality enhancements into media experiences. Such systems may be constrained in the capacity to support context-dependent content retrieval, intelligent layer composition, dimensionally responsive visualization, or adaptive modulation of sensory outputs. For example, many existing solutions may be unable to support responsive content transformations based on geographically dependent data, time-specific user states, or the evolving parameters of interactive engagement. Similarly, systems that rely on static content structures may be unable to reflect user participation, chain-of-use relationships, or multi-stage adaptive augmentation across user communities.

Limitations also exist in systems intended to support transactional, incentive-based, or privacy-preserving mechanisms for data contributions associated with personal, physiological, or contextual inputs. In many instances, known systems may lack the structural capability to manage secure valuation, access control, or dynamic representation of such data in a manner that aligns with user-defined privacy preferences, distributed data management strategies, or multi-party participation models. The collective challenges hinder the ability to provide fully responsive, adaptive, and immersive content experiences that meaningfully adjust to user context, preferences, environmental conditions, and multi-modal sensory channels.

Therefore, there is a need for improved methods, systems, apparatuses, and devices for providing content to viewers for enhancing wellbeing of the viewers that may overcome one or more of the preceding problems.

### SUMMARY OF DISCLOSURE

This summary is provided to introduce a selection of concepts in a simplified form, that are further described below in the Detailed Description. This summary is not intended to identify key features or essential features of the claimed subject matter. Nor is this summary intended to be used to limit the claimed subject matter's scope.

The present disclosure provides a method for providing content to viewers for enhancing health conditions of the viewers. Further, the method may include receiving, using a communication device, a media content from a broadcaster device. Further, the method may include receiving, using the communication device, one or more broadcaster preferences from the broadcaster device. Further, the one or more broadcaster preferences include one or more viewer context variables. Further, the method may include generating, using a processing device, an augmented media content for the enhancing of the health conditions of a viewer based on the one or more viewer context variables and the media content. Further, the augmented media content includes the media content and the one or more viewer context variables. Further, the augmented media content further includes two or more parameters corresponding to two or more augmented media contents. Further, the two or more parameters of the two or more augmented media contents may be configured to drive two or more sensory stimuli in the viewer. Further, the method may include transmitting, using the communication device, the augmented media content to a viewer device associated with the viewer. Further, the viewer device may be configured for presenting the augmented media content to the viewer. Further, the presenting of the augmented media content on the viewer device may be based on one or more viewer context values corresponding to the one or more viewer context variables. Further, the one or more viewer context variables correspond to one or more viewer sensors comprised in the viewer device. Further, the one or more viewer sensors may be configured for generating the one or more viewer context values for the one or more viewer context variables based on detecting a response of the viewer corresponding to the augmented media content. Further, the viewer device may be configured for generating two or more control signals based on the augmented media content. Further, the two or more control signals may include a first control signal, a second control signal, and a third control signal. Further, the viewer device further may include an audio output device which may be configured for generating a sound wave based on the first control signal to drive a first sensory stimulus in the viewer. Further, the viewer device further may include a visual output device which may be configured for generating a light based on the second control signal to drive a second sensory stimulus in the viewer. Further, the viewer device further may include a vibrotactile device which may be configured for generating a vibration based on the third control signal to drive a third sensory stimulus in the viewer. Further, the two or more sensory stimuli include the first sensory stimulus, the second sensory stimulus, and the third sensory stimulus. Further, the two or more sensory stimuli enhance the health conditions of the viewer. Further, the method may include transmitting, using the communication device, the augmented media content to the broadcaster device. Further, the method may include receiving, using the communication device, the one or more viewer context values corresponding to the one or more viewer context variables from the viewer device. Further, the method may include analyzing, using the processing device, the one or more viewer context values and the one or more viewer context variables corresponding to the one or more viewer context values using one or more first machine learning models. Further, the method may include generating, using the processing device, a health data for the viewer based on the analyzing of the one or more viewer context values. Further, the health data may include a health profile of the viewer. Further, the method may include storing, using a storage device, the health data of the viewer in a distributed ledger. Further, the method may include retrieving, using the storage device, two or more health data of two or more viewers. Further, the method may include analyzing, using the processing device, the two or more health data. Further, the method may include generating, using the processing device, two or more augmentation contents based on the analyzing of the two or more health data. Further, the method may include storing, using the storage device, the two or more augmentation contents in one or more databases.

The present disclosure provides a system for providing content to viewers for enhancing health conditions of the viewers. Further, the system may include a communication device. Further, the communication device may be configured for receiving a media content from a broadcaster device. Further, the communication device may be configured for receiving one or more broadcaster preferences from the broadcaster device. Further, the one or more broadcaster preferences include one or more viewer context variables. Further, the communication device may be configured for transmitting an augmented media content to a viewer device associated with a viewer. Further, the viewer device may be configured for presenting the augmented media content to the viewer. Further, the presenting of the augmented media content on the viewer device may be based on one or more viewer context values corresponding to the one or more viewer context variables. Further, the one or more viewer context variables correspond to one or more viewer sensors comprised in the viewer device. Further, the one or more viewer sensors may be configured for generating the one or more viewer context values for the one or more viewer context variables based on detecting a response of the viewer corresponding to the augmented media content. Further, the viewer device may be configured for generating two or more control signals based on the augmented media content. Further, the two or more control signals may include a first control signal, a second control signal, and a third control signal. Further, the viewer device further may include an audio output device which may be configured for generating a sound wave based on the first control signal to drive a first sensory stimulus in the viewer. Further, the viewer device further may include a visual output device which may be configured for generating a light based on the second control signal to drive a second sensory stimulus in the viewer. Further, the viewer device further may include a vibrotactile device which may be configured for generating a vibration based on the third control signal to drive a third sensory stimulus in the viewer. Further, the two or more sensory stimuli include the first sensory stimulus, the second sensory stimulus, and the third sensory stimulus. Further, two or more sensory stimuli enhance the health conditions of the viewer. Further, the communication device may be configured for transmitting the augmented media content to the broadcaster device. Further, the communication device may be configured for receiving the one or more viewer context values corresponding to the one or more viewer context variables from the viewer device. Further, the system may include a processing device communicatively coupled with the communication device. Further, the processing device may be configured for generating the augmented media content for the enhancing of the health conditions of a viewer based on the one or more viewer context variables and the media content. Further, the augmented media content includes the media content and the one or more viewer context variables. Further, the augmented media content further includes two or more parameters corresponding to two or more augmented media contents. Further, the two or more parameters of the two or more augmented media contents may be configured to drive the two or more sensory stimuli in the viewer. Further, the processing device may be configured for analyzing the one or more viewer context values and the one or more viewer context variables corresponding to the one or more viewer context values using one or more first machine learning models. Further, the processing device may be configured for generating a health data for the viewer based on the analyzing of the one or more viewer context values. Further, the health data may include a health profile of the viewer. Further, the processing device may be configured for analyzing two or more health data. Further, the processing device may be configured for generating two or more augmentation contents based on the analyzing of the two or more health data. Further, the system may include a storage device communicatively coupled with the processing device. Further, the storage device may be configured for storing the health data of the viewer in a distributed ledger. Further, the storage device may be configured for retrieving the two or more health data of two or more viewers. Further, the storage device may be configured for storing the two or more augmentation contents in one or more databases.

The present disclosure provides a device for providing content to viewers for enhancing health conditions of the viewers. Further, the device may include a communication unit. Further, the communication unit may be configured for receiving one or more media signals from a broadcaster device. Further, the one or more media signal corresponds to a media content. Further, the communication unit may be configured for receiving one or more broadcaster preferences from the broadcaster device. Further, the one or more broadcaster preferences include one or more viewer context variables. Further, the device may include a processing unit communicatively coupled with the communication unit. Further, the processing unit may be configured for generating an augmented media signal for the enhancing of the health conditions of a viewer based on the one or more media signals and the one or more broadcaster preferences. Further, the processing unit may be configured for generating two or more control signals based on the augmented media signal. Further, the device may include one or more visual output devices communicatively coupled with the processing unit. Further, the visual output device may be configured for generating a light based on a first control signal. Further, the two or more control signals include the first control signal. Further, the viewer may be associated with the one or more visual output devices. Further, the device may include one or more audio output devices communicatively coupled with the processing unit. Further, the one or more audio output devices may be configured for generating one or more sound waves based on a second control signal. Further, the two or more control signals further include the second control signal. Further, the viewer may be further associated with the one or more audio output devices. Further, the device may include one or more vibrotactile devices communicatively coupled with the processing unit. Further, the one or more vibrotactile devices may be configured for generating one or more vibrations based on a third control signal. Further, the two or more control signals further include the third control signal. Further, the viewer may be further associated with the one or more vibrotactile devices. Further, the light, the one or more sound waves, and the one or more vibrations enhance the health conditions of the viewer. Further, the device may include one or more sensor devices communicatively coupled with the processing unit. Further, the one or more sensor devices may be configured for detecting one or more responses of the viewer to one or more of the light, the one or more sound waves, and the one or more vibrations. Further, the one or more sensor devices may be configured for generating one or more viewer context values for the one or more viewer context variables based on the detecting of the one or more responses. Further, the generating of the augmented media signal for the enhancing of the health conditions may be further based on the one or more viewer context values.

Both the foregoing summary and the following detailed description provide examples and are explanatory only. Accordingly, the foregoing summary and the following detailed description should not be considered to be restrictive. Further, features or variations may be provided in addition to those set forth herein. For example, embodiments may be directed to various feature combinations and sub-combinations described in the detailed description.

### BRIEF DESCRIPTIONS OF DRAWINGS

The accompanying drawings, which are incorporated in and constitute a part of this disclosure, illustrate various embodiments of the present disclosure. The drawings contain representations of various trademarks and copyrights owned by the Applicants. In addition, the drawings may contain other marks owned by third parties and are being used for illustrative purposes only. All rights to various trademarks and copyrights represented herein, except those belonging to their respective owners, are vested in and the property of the applicants. The applicants retain and reserve all rights in their trademarks and copyrights included herein, and grant permission to reproduce the material only in connection with reproduction of the granted patent and for no other purpose.

Furthermore, the drawings may contain text or captions that may explain certain embodiments of the present disclosure. This text is included for illustrative, non-limiting, explanatory purposes of certain embodiments detailed in the present disclosure.

FIG. 1 is an illustration of an online platform consistent with various embodiments of the present disclosure.

FIG. 2 illustrates a flowchart of a method of generating an augmented media content, in accordance with some embodiments.

FIG. 3 illustrates a flowchart of a method of generating an augmented media content including a depiction of user interfaces presented on a broadcaster device and a viewer device, in accordance with some embodiments.

FIG. 4 illustrates embedding of augmentation content in a media content for generating an augmented media content, in accordance with some embodiments.

FIG. 5 illustrates an exemplary user interface presentable on a broadcaster device for facilitating augmentation of media content, in accordance with some embodiments.

FIG. 6 illustrates augmentation of media content based on viewing region (as characterized by zoom factor, distance, etc.) in accordance with some embodiments.

FIG. 7 illustrates a flowchart of a method of generating augmented media content, in accordance with some embodiments.

FIG. 8 illustrates a flowchart of a method of generating augmented media content based on analysis of media content, in accordance with some embodiments.

FIG. 9 is a block diagram of a system for providing content to viewers for enhancing wellbeing of the viewers, in accordance with some embodiments.

FIG. 10 is a block diagram of the system for providing the content to the viewers for enhancing wellbeing of the viewers, in accordance with some embodiments.

FIG. 11 is a flowchart of a method for providing content to viewers for enhancing wellbeing of the viewers, in accordance with some embodiments.

FIG. 12 is a flowchart of a method for embedding augmentation content in media content for providing the content to the viewers for enhancing the wellbeing of the viewers, in accordance with some embodiments.

FIG. 13 is a flowchart of a method for generating health data for the viewers for providing the content to the viewers, in accordance with some embodiments.

FIG. 14 is a flowchart of a method for acquiring the health data from the viewers for providing the content to the viewers, in accordance with some embodiments.

FIG. 15 is a flowchart of a method for generating augmentation content for providing the content to the viewers, in accordance with some embodiments.

FIG. 16 is a flowchart of a method for determining requirements of the viewers for providing the content to the viewers, in accordance with some embodiments.

FIG. 17 is a block diagram of a computing device for implementing the methods disclosed herein, in accordance with some embodiments.

FIG. 18 is a flow diagram of a method for algorithmically generating personalized content by preset relationships for providing content to viewers for enhancing wellbeing of the viewers, in accordance with some embodiments.

FIG. 19 is a visual representation of traveling of signals into a dimensional space for facilitating generating of the content for viewers for enhancing wellbeing of the viewers, in accordance with some embodiments.

FIG. 20A illustrates a flowchart of a method 2000 for providing content to viewers for enhancing health conditions of the viewers, in accordance with some embodiments.

FIG. 20B illustrates a continuation of the flowchart of the method 2000 for providing content to viewers for enhancing health conditions of the viewers, in accordance with some embodiments.

FIG. 20C illustrates a continuation of the flowchart of the method 2000 for providing content to viewers for enhancing health conditions of the viewers, in accordance with some embodiments.

FIG. 20D illustrates a continuation of the flowchart of the method 2000 for providing content to viewers for enhancing health conditions of the viewers, in accordance with some embodiments.

FIG. 21 illustrates a flowchart of a method 2100 for providing content to viewers for enhancing health conditions of the viewers including determining, using the processing device 904, at least one requirement of the viewer for the enhancing of the health conditions of the viewer and the at least one viewer context variable corresponding to the at least one requirement, in accordance with some embodiments.

FIG. 22 illustrates a flowchart of a method 2200 for providing content to viewers for enhancing health conditions of the viewers including computing, using the processing device 2404, a phrase-state correlation between the at least one viewer state value and each of the plurality of content phrases, in accordance with some embodiments.

FIG. 23 illustrates a flowchart of a method 2300 for providing content to viewers for enhancing health conditions of the viewers including generating, using the processing device 2404, a cryptographic audit record for the augmented media content, in accordance with some embodiments.

FIG. 24 illustrates a block diagram of a system 2400 for providing content to viewers for enhancing health conditions of the viewers, in accordance with some embodiments.

FIG. 25 illustrates a block diagram of the system 2400 for providing content to viewers for enhancing health conditions of the viewers, in accordance with some embodiments.

FIG. 26 illustrates a block diagram of a device 2500 for providing content to viewers for enhancing health conditions of the viewers, in accordance with some embodiments.

FIG. 27 illustrates a therapeutic application of the device 2500 for providing content to viewers for enhancing health conditions of the viewers, in accordance with some embodiments.

### DETAILED DESCRIPTION OF DISCLOSURE

As a preliminary matter, it will readily be understood by one having ordinary skill in the relevant art that the present disclosure has broad utility and application. As should be understood, any embodiment may incorporate only one or a plurality of the above-disclosed aspects of the disclosure and may further incorporate only one or a plurality of the above-disclosed features. Furthermore, any embodiment discussed and identified as being “preferred” is considered to be part of a best mode contemplated for carrying out the embodiments of the present disclosure. Other embodiments also may be discussed for additional illustrative purposes in providing a full and enabling disclosure. Moreover, many embodiments, such as adaptations, variations, modifications, and equivalent arrangements, will be implicitly disclosed by the embodiments described herein and fall within the scope of the present disclosure.

Accordingly, while embodiments are described herein in detail in relation to one or more embodiments, it is to be understood that this disclosure is illustrative and exemplary of the present disclosure, and are made merely for the purposes of providing a full and enabling disclosure. The detailed disclosure herein of one or more embodiments is not intended, nor is to be construed, to limit the scope of patent protection afforded in any claim of a patent issuing here from, which scope is to be defined by the claims and the equivalents thereof. It is not intended that the scope of patent protection be defined by reading into any claim limitation found herein and/or issuing here from that does not explicitly appear in the claim itself.

Thus, for example, any sequence(s) and/or temporal order of steps of various processes or methods that are described herein are illustrative and not restrictive. Accordingly, it should be understood that, although steps of various processes or methods may be shown and described as being in a sequence or temporal order, the steps of any such processes or methods are not limited to being carried out in any particular sequence or order, absent an indication otherwise. Indeed, the steps in such processes or methods generally may be carried out in various different sequences and orders while still falling within the scope of the present disclosure. Accordingly, it is intended that the scope of patent protection is to be defined by the issued claim(s) rather than the description set forth herein.

Additionally, it is important to note that each term used herein refers to that which an ordinary artisan would understand such term to mean based on the contextual use of such term herein. To the extent that the meaning of a term used herein—as understood by the ordinary artisan based on the contextual use of such term—differs in any way from any particular dictionary definition of such term, it is intended that the meaning of the term as understood by the ordinary artisan should prevail.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items of the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the claims found herein and/or issuing here from. The present disclosure contains headers. It should be understood that these headers are used as references and are not to be construed as limiting upon the subjected matter disclosed under the header.

The present disclosure includes many aspects and features. Moreover, while many aspects and features relate to, and are described in the context of the disclosed use cases, embodiments of the present disclosure are not limited to use only in this context.

In general, the method disclosed herein may be performed by one or more computing devices. For example, in some embodiments, the method may be performed by a server computer in communication with one or more client devices over a communication network such as, for example, the Internet. In some other embodiments, the method may be performed by one or more of at least one server computer, at least one client device, at least one network device, at least one sensor and at least one actuator. Examples of the one or more client devices and/or the server computer may include, a desktop computer, a laptop computer, a tablet computer, a personal digital assistant, a portable electronic device, a wearable computer, a smart phone, an Internet of Things (IoT) device, a smart electrical appliance, a video game console, a rack server, a super-computer, a mainframe computer, mini-computer, micro-computer, a storage server, an application server (e.g. a mail server, a web server, a real-time communication server, an FTP server, a virtual server, a proxy server, a DNS server etc.), a quantum computer, and so on. Further, one or more client devices and/or the server computer may be configured for executing a software application such as, for example, but not limited to, an operating system (e.g. Windows, Mac OS, Unix, Linux, Android, etc.) in order to provide a user interface (e.g. GUI, touch-screen based interface, voice based interface, gesture based interface etc.) for use by the one or more users and/or a network interface for communicating with other devices over a communication network. Accordingly, the server computer may include a processing device configured for performing data processing tasks such as, for example, but not limited to, analyzing, identifying, determining, generating, transforming, calculating, computing, compressing, decompressing, encrypting, decrypting, scrambling, splitting, merging, interpolating, extrapolating, redacting, anonymizing, encoding and decoding. Further, the server computer may include a communication device configured for communicating with one or more external devices. The one or more external devices may include, for example, but are not limited to, a client device, a third party database, public database, a private database and so on. Further, the communication device may be configured for communicating with the one or more external devices over one or more communication channels. Further, the one or more communication channels may include a wireless communication channel and/or a wired communication channel. Accordingly, the communication device may be configured for performing one or more of transmitting and receiving of information in electronic form. Further, the server computer may include a storage device configured for performing data storage and/or data retrieval operations. In general, the storage device may be configured for providing reliable storage of digital information. Accordingly, in some embodiments, the storage device may be based on technologies such as, but not limited to, data compression, data backup, data redundancy, deduplication, error correction, data finger-printing, role based access control, and so on.

Further, one or more steps of the method disclosed herein may be initiated, maintained, controlled and/or terminated based on a control input received from one or more devices operated by one or more users such as, for example, but not limited to, an end user, an admin, a service provider, a service consumer, an agent, a broker and a representative thereof. Further, the user as defined herein may refer to a human, an animal or an artificially intelligent being in any state of existence, unless stated otherwise, elsewhere in the present disclosure. Further, in some embodiments, the one or more users may be required to successfully perform authentication in order for the control input to be effective. In general, a user of the one or more users may perform authentication based on the possession of a secret human readable secret data (e.g. username, password, passphrase, PIN, secret question, secret answer etc.) and/or possession of a machine readable secret data (e.g. encryption key, decryption key, bar codes, etc.) and/or or possession of one or more embodied characteristics unique to the user (e.g. biometric variables such as, but not limited to, fingerprint, palm-print, voice characteristics, behavioral characteristics, facial features, iris pattern, heart rate variability, evoked potentials, brain waves, and so on) and/or possession of a unique device (e.g. a device with a unique physical and/or chemical and/or biological characteristic, a hardware device with a unique serial number, a network device with a unique IP/MAC address, a telephone with a unique phone number, a smartcard with an authentication token stored thereupon, etc.). Accordingly, the one or more steps of the method may include communicating (e.g. transmitting and/or receiving) with one or more sensor devices and/or one or more actuators in order to perform authentication. For example, the one or more steps may include receiving, using the communication device, the secret human readable data from an input device such as, for example, a keyboard, a keypad, a touch-screen, a microphone, a camera and so on. Likewise, the one or more steps may include receiving, using the communication device, the one or more embodied characteristics from one or more biometric sensors.

Further, one or more steps of the method may be automatically initiated, maintained and/or terminated based on one or more predefined conditions. In an instance, the one or more predefined conditions may be based on one or more contextual variables. In general, the one or more contextual variables may represent a condition relevant to the performance of the one or more steps of the method. The one or more contextual variables may include, for example, but are not limited to, location, time, identity of a user associated with a device (e.g. the server computer, a client device etc.) corresponding to the performance of the one or more steps, environmental variables (e.g. temperature, humidity, pressure, wind speed, lighting, sound, etc.) associated with a device corresponding to the performance of the one or more steps, physical state and/or physiological state and/or psychological state of the user, physical state (e.g. motion, direction of motion, orientation, speed, velocity, acceleration, trajectory, etc.) of the device corresponding to the performance of the one or more steps and/or semantic content of data associated with the one or more users. Accordingly, the one or more steps may include communicating with one or more sensors and/or one or more actuators associated with the one or more contextual variables. For example, the one or more sensors may include, but are not limited to, a timing device (e.g. a real-time clock), a location sensor (e.g. a GPS receiver, a GLONASS receiver, an indoor location sensor etc.), a biometric sensor (e.g. a fingerprint sensor), an environmental variable sensor (e.g. temperature sensor, humidity sensor, pressure sensor, etc.) and a device state sensor (e.g. a power sensor, a voltage/current sensor, a switch-state sensor, a usage sensor, etc. associated with the device corresponding to performance of the or more steps).

Further, the one or more steps of the method may be performed one or more number of times. Additionally, the one or more steps may be performed in any order other than as exemplarily disclosed herein, unless explicitly stated otherwise, elsewhere in the present disclosure. Further, two or more steps of the one or more steps may, in some embodiments, be simultaneously performed, at least in part. Further, in some embodiments, there may be one or more time gaps between performance of any two steps of the one or more steps.

Further, in some embodiments, the one or more predefined conditions may be specified by the one or more users. Accordingly, the one or more steps may include receiving, using the communication device, the one or more predefined conditions from one or more and devices operated by the one or more users. Further, the one or more predefined conditions may be stored in the storage device. Alternatively, and/or additionally, in some embodiments, the one or more predefined conditions may be automatically determined, using the processing device, based on historical data corresponding to performance of the one or more steps. For example, the historical data may be collected, using the storage device, from a plurality of instances of performance of the method. Such historical data may include performance actions (e.g. initiating, maintaining, interrupting, terminating, etc.) of the one or more steps and/or the one or more contextual variables associated therewith. Further, machine learning may be performed on the historical data in order to determine the one or more predefined conditions. For instance, machine learning on the historical data may determine a correlation between one or more contextual variables and performance of the one or more steps of the method. Accordingly, the one or more predefined conditions may be generated, using the processing device, based on the correlation.

Further, one or more steps of the method may be performed at one or more spatial locations. For instance, the method may be performed by a plurality of devices interconnected through a communication network. Accordingly, in an example, one or more steps of the method may be performed by a server computer. Similarly, one or more steps of the method may be performed by a client computer. Likewise, one or more steps of the method may be performed by an intermediate entity such as, for example, a proxy server. For instance, one or more steps of the method may be performed in a distributed fashion across the plurality of devices in order to meet one or more objectives. For example, one objective may be to provide load balancing between two or more devices. Another objective may be to restrict a location of one or more of an input data, an output data and any intermediate data there between corresponding to one or more steps of the method. For example, in a client-server environment, sensitive data corresponding to a user may not be allowed to be transmitted to the server computer. Accordingly, one or more steps of the method operating on the sensitive data and/or a derivative thereof may be performed at the client device.

### Overview

The present disclosure describes methods, systems, apparatuses, and devices for providing content to viewers for enhancing wellbeing of the viewers. Further, the present disclosure describes utilizing vibrational health records which take biometric and API data and store it in different methods including blockchain in order to determine hardware programming and generative content for health, therapy, or entertainment in mixed reality and media on any device. Further, the present disclosure utilizes machine learning and AI combined with active user data, trends, historical preferences, and existing databases.

Further, the present disclosure describes capturing biometrics and programmatically developing direct biofeedback treatments using audio, visual, and haptic/vibrotactile hardware and software, other sensory devices, implant devices, wearable devices, etc. Further, the present disclosure describes AI, related as the contextualization and processing, decision making neural net, and personal response of viewers. Further, the present disclosure describes the vibrational health record which is generated from the biometrics and preferences or external data sources on the blockchain.

Consistent with embodiments of the present disclosure, an online platform (also referred to herein as “platform” or media augmentation platform) for facilitating augmentation of media content, such as, for example, video, audio, multimedia, Virtual Reality (VR) content, etc. may be provided. This overview is provided to introduce a selection of concepts in a simplified form that are further described below. This overview is not intended to identify key features or essential features of the claimed subject matter. Nor is this overview intended to be used to limit the claimed subject matter's scope. The online platform may be used by individuals or companies to facilitate augmentation of media content.

In some embodiments, the present disclosure provides a live and prerecorded contextual video engagement system, which pulls in all available data from web or open frequency sources to allow a user to annotate and augment their own content in real-time.

If surrounding information can be used for an annotation, it is placed into the user's video editing dashboard in real-time. Known as Augmented Snapshot, these items can either be inserted into the timeline or on top of live or pre-recorded content. If the augmented content the user would like to use is not available, the user can insert a query or conditional statement. Therefore, when the content is viewed it pulls the augmented content most relevant to that viewer as designated by the broadcaster. This includes, but is not limited to: connected devices, supplemental media, personal and user information, location, time, trends, tags, social media, and e-commerce listings.

Accordingly, in an instance, when a snapshot is taken, an encoding server breaks down each input data comprising video into frames and utilizes local and cloud server processes along with a layer of API connections to analyze each frame for possible interactive elements. Further, taking the snapshot may include capturing a moment in time or a length of time from the input data. The broadcaster can use either a manual or automated process to place tracking and interaction points on people or items. The automated process connects with any data source which can provide relevant data for interaction points. This includes, but is not limited to, facial and image recognition databases, health databases, heat mapping, data from web crawlers, ad networks, content publishers comprising music or video streaming, feeds, plugins, tags, promotions, user and device data, or environmental and GPS data sources. Administrators, broadcasters and viewers can add and toggle the augmented viewing modes which are most useful to them. Results from these processes are used to insert the most logical interaction points and preprogrammed or generative content for augmented content, sign up options, relevant locations, social media, purchasing opportunities and other user data. This information is filtered for use by analyzing the metadata and preferences of the broadcaster, using that as a live query to pull in more relevant data from the contextual and user input systems. The control of this data is done with the System and User filters and the Snapshot.

Through the present invention all interactive elements are stored in a transmittable, digital interactive content container. Each container may have interaction points defined by the broadcaster or the viewer as determined by the decoding server. Viewers can utilize the available synchronized generative and aggregated data (such as live Augmented Reality (AR) data) to create their own snapshot additions to the content using interactive hotspots, input data, and media streams, they create.

For live broadcast, information and category prompts for the AR broadcast display will be selected ahead of time and can be triggered by the broadcaster or set ahead of time. These elements will be received by the viewer, then searched or filtered based on their interests. Interactive containers can be sent as links, embedded on pages, assigned to specified actions, activated by context or broadcasted across a large network of publishers. Viewer modes and filters are made available based on the contextually aware data coming from the APIs and information sources.

These viewing filters/modes may be switched between, showing only the relevant interactivity. The switching process is similar to how one may switch between video and camera mode on an iPhone. To provide the fastest and most optimum experience, the default viewing mode is based on the commonalities between the broadcaster's and viewer's preferences.

All activity is cataloged and cookied for transparent user history data controls. At any point, the viewer can see what information has been stored about them and their patterns. In real-time, they can change, add to, or remove this preference data to create more accurate recommendations and profiles for their ongoing experience. The system constantly crawls available data sources, identifies interactive file types and information, then presents new interactive options to both the broadcasters and viewer.

As advertisers and content creators place their promotions on the system, viewer activity and preferences create an unprecedented transparency and accuracy allow for true utility in the promotional process. This personalization allows for the system to provide contextually aware auto-response messages, data and coupons back to the viewer. The broadcaster can customize the graphics and number of redemptions of each coupon they create on the dashboard. This system is a self-contained, hyper-intelligent content creation, management, marketing and media platform which dramatically minimizes the number of clicks required to engage each end viewer. The broadcaster also has access to the most data available; viewing patterns, hot spots, mouse movements, social trends, social graphs, opt-ins and interaction history. The recommendation system works for both broadcaster and viewer, helping to determine the most effective interactions and methods to communicate the intended message. This system takes advantage of as many emerging technologies and data sources possible in order to deliver the most personalized and real-time, interactive experience available.

Further, the present disclosure describes a first method of providing augmented media. The first method may include receiving, using a communication unit, a media content from a broadcaster device. Further, the first method may include receiving, using the communication unit, at least one broadcaster preference from the broadcaster device. Further, the broadcaster preference may include at least one viewer context variable. Further, the first method may include retrieving, using the communication unit, a plurality of augmentation content from at least one database based on the at least one broadcaster preference. Further, the first method may include transmitting, using the communication unit, the plurality of augmentation content to the broadcaster device. Further, the broadcaster device may be configured to present the plurality of augmentation content. Further, the first method may include receiving, using the communication unit, a selection of at least one augmentation content from the broadcaster device. Further, the first method may include embedding, using a processing unit, each of the at least one augmentation content and the at least one viewer context variable in the media content to obtain an augmented media content. Further, presenting of the augmented media content on a viewer device may be based on at least one viewer context value corresponding to the at least one viewer context variable. Further, the at least one viewer context value may be associated with the viewer device. Further, the first method may include transmitting, using the communication unit, the augmented media content to the broadcaster device.

Further, the present disclosure describes a second method of providing augmented media content. The second method may include receiving, using the processing unit, at least one broadcaster preference. Further, the second method may include receiving, using a processing unit, a media content from a media source. Further, the second method may include analyzing, using the processing unit, the media content. Further, the second method may include identifying, using the processing unit, at least one interaction element in the media content based on the analyzing. Further, the second method may include identifying, using the processing unit, at least one augmentation content based on each of the at least one interaction element and the at least one broadcaster preference. Further, the second method may include embedding, using the processing unit, the at least one augmentation content in the media content to obtain an augmented media content. Further, the second method may include transmitting, using a communication unit, the augmented media content to a viewer device. Further, the viewer device may be configured to present the media content. Further, the viewer device may be associated with at least one viewer preference and at least one viewer contextual data. Further, presenting of the at least one augmentation content may be based on the at least one viewer preference and the at least one viewer contextual data.

Further, the present disclosure describes a third method of providing augmented media content. The third method may include receiving, using a communication unit, a media content from a broadcaster device. Further, the third method may include receiving, using the communication unit, at least one broadcaster contextual data from the broadcaster device. Further, the at least one broadcaster contextual data may be associated with one or more of the media content and a broadcaster associated with the user device. Further, the third method may include analyzing, using a processing unit, one or more of the media content and the at least one broadcaster contextual data. Further, the third method may include identifying, using the processing unit, a plurality of augmentation content based on the analyzing. Further, the third method may include transmitting, using the communication unit, the plurality of augmentation content to the broadcaster device. Further, the third method may include receiving, using the communication unit, a selection of at least one augmentation content from the plurality of augmentation content. Further, the third method may include embedding, using the processing, one or more of the at least one augmentation content in the media content to obtain an augmented media content. Further, the third method may include transmitting, using the communication unit, the augmented media content to the broadcaster device.

Further, the present disclosure describes a system for providing augmented media. The system may include a communication unit configured for receiving a media content from a broadcaster device. Further, the communication unit may be configured for receiving at least one broadcaster preference from the broadcaster device. Further, the broadcaster preference may include at least one viewer context variable. Further, the communication unit may be configured for retrieving a plurality of augmentation content from at least one database based on the at least one broadcaster preference. Further, the communication unit may be configured for transmitting the plurality of augmentation content to the broadcaster device. Further, the broadcaster device may be configured to present the plurality of augmentation content. Further, the system may include receiving a selection of at least one augmentation content from the broadcaster device. Further, the system may include transmitting the augmented media content to the broadcaster device. Further, the system may include a processing unit configured for embedding each of the at least one augmentation content and the at least one viewer context variable in the media content to obtain an augmented media content. Further, presenting of the augmented media content on a viewer device may be based on at least one viewer context value corresponding to the at least one viewer context variable. Further, the at least one viewer context value may be associated with the viewer device.

Further, the present disclosure describes a software application that intakes signals and splits them into the different outputs of light, sound, and vibration which are defined as having a programmable pulse width, amplitudes, frequencies, and modulation depths in addition to the standard music player functions. It saves these to a read/write format (.json). Further, the software synchronizes all these elements based on the ideal wellness program and provides seamless integration of music combined with the frequencies to a proprietary file format (.JSON+MUSIC FILE=.LSV). Further, the proprietary file format is a programmatic sequencing of live or predetermined oscillators combined with timed music and visuals in a proprietary way. Further, the signals may be included the media content transmitted from a broadcaster device associated with a broadcaster. Further, the broadcaster device 1002 may be associated with a broadcaster. Further, the broadcaster may be an artificial intelligence (AI) entity. Further, the augmented media content which is generated has the proprietary file format (.LSV).

As used herein, visual output may be delivered through one or more visual presentation mechanisms, including but not limited to screen-based displays, projected imagery, head-mounted or wearable displays, ambient lighting systems, or temporally modulated or stroboscopic light sources. In some embodiments, visual output comprises screen-rendered visual content such as images, animations, video, generative graphics, color fields, geometric patterns, symbolic imagery, or immersive virtual, augmented, or mixed-reality environments. In other embodiments, visual output comprises temporally modulated illumination, including flicker-based or stroboscopic light delivered via LEDs, light panels, architectural lighting, or embedded light emitters, wherein such illumination may be modulated according to one or more parameters including frequency, duty cycle, intensity, phase, waveform, spatial distribution, or synchronization relative to audio and haptic or vibroacoustic outputs. Screen-based visual content and temporally modulated or stroboscopic light may be used independently or concurrently and may be dynamically adjusted based on detected viewer state, safety constraints, or feedback signals.

### Deficit—Reduction Protocols:

| Protocol | Multi-Modal Specification |
|---|---|
| Anxiety Reduction | Audio: Synchronized low-frequency (theta range 4-8 Hz) \| |
| | Visual: Scaling 4-8 Hz stroboscopic light\| Haptic: Rhythmic |
| | vibrotactile sinewaves at synchronized, decreasing tempo |
| Sleep Induction | Audio: Binaural beats at delta frequencies (0.5-4 Hz) \| Visual: |
| | Delta strobe with gradually dimming light \| Haptic: Decelerating |
| | cadence synchronized with target respiratory rate |
| Pain Management | Audio: Entrainment at alpha frequencies (8-12 Hz) \| Visual: |
| | Distraction-optimized content \| Haptic: Gate-control stimulation |
| | at affected body regions |
| Trauma Processing | Audio: Bilateral stimulation alternating L/R channels \| Visual: |
| | Safe-state anchors, grounding sequences with Alpha to Theta |
| | strobe \| Haptic: Bilateral alternating patterns resonant with sound |
| Stress Reduction | Audio: Parasympathetic-activating frequencies \| Visual: Nature- |
| | based content with Theta to Delta strobe. \| Haptic: Coherent |
| | breathing guidance synchronized across vibration channels |

### Positive-State Enhancement Protocols:

| Protocol | Multi-Modal Specification |
|---|---|
| Happiness | Audio: Major-key at elevated tempo (100-130 BPM) \| Visual: |
| Elevation | Reward-associated content in \| Haptic: Pleasure-linked patterns |
| | calibrated to viewer musical preference history |
| Joy Amplification | Audio: Crescendo patterns building to euphoric release \| Visual: |
| | Peak-experience sequences \| Haptic: Synchronized, excitatory |
| | vibrational feedback connected to audio |
| Gratitude | Audio: Heart-coherence guided entrainment \| Visual: Memory- |
| Cultivation | associated cues personalized to viewer \| Haptic: Chest and center |
| | mass focused |
| Motivation | Audio: Energizing frequencies in beta range (12-30 Hz) \| Visual: |
| Enhancement | Goal-visualization content \| Haptic: Activating sequences with |
| | increasing intensity |
| Creativity | Audio: Alpha-wave entrainment (8-12 Hz) \| Visual: Novel- |
| Stimulation | association patterns \| Haptic: Exploratory sequences with varied |
| | textures of stimulating sound enhancing the audio entrainment |
| Social Connection | Audio: Prosocial cues with other users \| Visual: Connectedness- |
| | associated content patterns \| Haptic: Unified group sensations |
| Flow State | Audio: Focus-enhancing with alpha bilateral oscillations \| |
| Induction | Visual: Challenge-matched content progression \| Haptic: |
| | Synchronous panning |
| Resilience Building | Audio: Confidence-associated patterns \| Visual: Mastery- |
| | reinforcing sequences showing progressive achievement \| Haptic: |
| | Grounding feedback in lower body |

Further, the present disclosure describes a hardware device that takes analog/digital signals and split them into the various output channels in a synchronized way.

In some embodiments, the present disclosure relates to systems, methods, platforms, devices, and machine-implemented frameworks for generating, augmenting, transmitting, and adaptively transforming media content in order to enhance wellbeing, engagement, therapeutic outcomes, entertainment value, and contextual personalization for viewers. In some embodiments, the system may include a communication device, a processing device, a set of layered augmentation engines, a plurality of viewer sensors, a broadcaster interface, a dynamically configurable software environment, and an optional distributed ledger for secure storage and valuation of biometric, contextual, or augmentation-related data.

In some embodiments, the technology may support the generation of augmented media content comprising audio, video, haptic, vibrotactile, extended-reality layers, biometric-responsive elements, and other sensory or computational modalities.

In some embodiments, the system may be used to embed, within a media content item, one or more viewer context variables derived from a sensor set integrated within a viewer device, where such sensors may include optical sensors, acoustic sensors, motion sensors, physiological sensors, biological sensors, biokinetic sensors, or implantable devices capable of capturing a viewer's physical, physiological, emotional, psychological, or behavioral responses in real time.

In some embodiments, the system may implement machine learning models, generative algorithms, or a hybrid combination thereof to compute augmentation layers, user-state inferences, predictive personalizations, and dynamic transformations to media content.

In some embodiments, the present disclosure may enable a broadcaster, who may be an AI entity or a human user, to supply media content, preferences, and augmentation parameters, while the system may retrieve augmentation content from one or more databases, embed such augmentation content into the media content, and deliver a final composite media stream to a viewer device, possibly in real time and possibly in repeated peer-to-peer augmentation cycles.

In some embodiments, the system may manipulate multi-modal signals from numerous digital and analog channels, including light, sound, vibration, haptic, scalar, radio, Zigbee, Bluetooth, WiFi, LiFi, IR, projection, VR/XR content, pro-audio channels, and other network-connected devices.

In some embodiments, the system may introduce an inherent technical improvement to media processing technology by enabling real-time contextual augmentation of live or prerecorded media content using external APIs, data feeds, time-encoded interactions, geolocation inputs, social or trend information, and sensor-derived contextual values. A technical problem addressed by the improvement is the inability of conventional video or multimedia editing tools to adaptively integrate live contextual data into content at frame-level granularity. Existing systems typically rely on static timelines or offline processing, which may not responsively incorporate data streams that reflect the current state of a viewer, environment, or broadcaster.

In some embodiments, the system may solve the problem by providing a real-time augmentation engine that breaks incoming media streams into frames, identifies interaction points through object recognition or facial recognition, and retrieves augmentation objects from cloud services or databases based on the interaction points. In some embodiments, an encoding server may automatically process each frame using machine learning-based classifiers or rule-based interactions to detect opportunities for augmented snapshots, annotations, overlay objects, or viewer-specific content items.

In some embodiments, the above improvement enhances the technology of media editing and broadcasting by enabling fine-grained, live transformations that were not previously possible at a commercial scale. In some embodiments, different implementation paths may include using edge-side processing for latency-sensitive augmentation, cloud-side processing for heavy generative computations, or hybrid processing that distributes tasks across multi-node infrastructures for load balancing.

In some embodiments, the system may improve augmented-reality rendering by enabling dynamically adjustable viewing regions based on zoom level, gaze-tracking inputs, spatial positioning, or temporal subsections of the media being consumed. A technical problem addressed here is the inability of conventional AR systems to adjust contextual overlays dynamically based on the viewer's focal zone or zoom magnification.

In some embodiments, the system may track zoom factor, field-of-view parameters, sensor-based gaze vectors, and temporal intervals to determine the appropriate subset of augmentation content to present. For example, if a viewer zooms from 1× to 10×, the system may automatically expand the contextual area displayed, retrieving and placing augmentation content relevant to a broader spatial region or temporal frame. The above improvement may improve AR technology by allowing context-aware, multi-scale augmentation rendering. In some embodiments, implementations may rely on computer vision-based gaze detection, viewer sensor-based motion capture, SLAM (Simultaneous Localization and Mapping), or AI-assisted region classification.

In some embodiments, the present disclosure improves multi-modal hardware control by programmatically splitting analog and digital signals into synchronized channels of light, sound, vibration, and haptic outputs, including programmable frequencies, amplitudes, pulse widths, phases, tones, beats per minute, modulation depths, prilling patterns, cymatic patterns, and other oscillatory characteristics. A technical problem addressed is that conventional multimedia systems may not unify cross-modal outputs with therapeutic precision or dynamically adapt output across heterogeneous devices.

In some embodiments, the solution may involve generating a proprietary LSV file format that links oscillatory definitions to music tracks and visual layers, combining predetermined or generative oscillators with timed sequences. In some embodiments, the system may perform real-time output channel allocation, buffer synchronization, clock alignment, or latency compensation across distributed devices, including home devices, vehicle-mounted devices, VR systems, and wearable haptics. The proprietary file format may improve hardware-software integration technology by enabling portable, high-resolution, cross-modal signal orchestration.

In some embodiments, the system may incorporate machine learning models for analyzing viewer data to determine wellness requirements, select augmentation content, and compute viewer context variables in real time. A technical problem addressed is the inability of traditional media platforms to adaptively personalize content based on physiological, emotional, or psychological context. In some embodiments, the system may solve the above problem by using one or more machine learning models, including generative models, predictive classifiers, or contextually aware neural networks, to infer states from biometric signals, behavioral interactions, historical preferences, or relational data. In some embodiments, the system may improve personalized content delivery technology by enabling algorithmic personalization loops where user responses feed back into the system to produce successive layers of personalized augmentation across single or synchronized multi-channel outputs.

In some embodiments, the system may improve blockchain and distributed ledger technology by storing biometric records, relational datasets, and viewer context variables in a ledger, and by assigning dynamically computed token values to such records. A technical problem addressed is that conventional healthcare data systems lack automated valuation mechanisms for biometric streams and do not support direct economic incentive models for data provision.

In some embodiments, the system may solve the above problem by enabling minting, transmitting, or monetizing cryptocurrency-like tokens that may represent viewer-contributed biometric data. In some embodiments, the system may determine token value using machine learning-based value assessment, usage frequency, health impact metrics, or statistical rarity of the underlying biometric dataset, improving distributed data valuation technologies and expanding blockchain functionality beyond conventional financial transactions to dynamic health-data-driven incentive models.

In some embodiments, the system may introduce a multi-dimensional waveform representation space, wherein signals may be rendered as extruding, deforming, or animating constructs with geometric properties such as vertices, polygons, torsion, rotation, cymatics, amplitude fields, or prilling behaviors. A technical problem addressed is the poor compatibility between traditional media timelines and the mathematical representation of complex therapeutic or XR stimuli. In some embodiments, the solution may involve representing all stimuli as evolving waveforms mapped into a vectored spatial domain, enabling generative algorithms to manipulate multidimensional oscillatory forms in real time. The above improvement may improve XR and signal-processing technologies by providing a unified geometric framework for multimodal generative content.

In some embodiments, the system may allow viewers to become broadcasters in a recursive cycle, such that each augmented media content item may be further augmented by downstream users. A technical problem addressed is that conventional media systems do not allow chained augmentation while preserving context variables. In some embodiments, the solution may include embedding viewer context variables, augmentation metadata, and chain-of-custody blockchain entries into each augmented media object. The above solution may improve collaborative media editing technology by enabling multi-party contextual augmentation across a distributed peer-to-peer network.

In some embodiments, the system may include conditional logic triggers that retrieve augmentation objects when certain dynamic conditions are satisfied, such as weather changes, geofence crossings, user-specified rules, or relational database triggers. The conditional logic triggers may improve conditional automation technology by enabling parameter-driven augmentation retrieval operations that conventional video editors cannot perform.

In some embodiments, the present disclosure describes a dedicated hardware device for receiving analog or digital signals and splitting them into synchronized output channels for light, sound, haptics, vibration, and biofeedback systems. A technical problem addressed is the lack of general-purpose consumer devices that may uniformly process multi-modal therapeutic oscillations across heterogeneous output devices.

In some embodiments, implementations may incorporate FPGA-based controllers, DSP modules, neural-network-accelerated oscillation generators, or adaptive analog-digital conversion layers to ensure synchronized, programmable cross-modal outputs. The FPGA-based controllers improve the technology of wellness-oriented output devices by offering generalized multi-channel signal control architecture.

In some embodiments, the system may expand the notion of cryptocurrency into a broader class of tokenized digital assets, including fungible tokens, non-fungible tokens, fractional tokens, utility tokens, governance tokens, derivative health-value tokens, biometric-performance tokens, and time-bounded incentive tokens. In some embodiments, such tokens may be minted based on biometric state changes, viewer engagement metrics, or contextual state data. The above feature may improve digital economy technologies by enabling health-responsive, context-aware, and programmatically generated token ecosystems that interact with wellness content generation and provide transparency.

In some embodiments, the hardware device may incorporate multi-port output channels, microcontroller-based timing circuits, DSP pipelines, and programmable waveform synthesizers. In some embodiments, the device may accept LSV file inputs, which may encode music tracks, oscillatory definitions, light sequences, geometric patterns, or vibration mappings. In some embodiments, the device may synchronize oscillatory characteristics across distributed output nodes using wireless protocols such as WiFi, Bluetooth LE, Zigbee, LiFi, near-infrared, or long-range mesh technologies.

In some embodiments, the hardware device may include embedded biometric sensors for reading user responses, thereby forming a closed feedback loop between input stimuli and output modulation, facilitating wellness-driven therapeutic control. Further, the integration of the embedded biometric sensors improves device-level oscillatory control technology by offering modular input-output reconfiguration and programmable sensory sequencing.

In some embodiments, the term “cryptocurrency” may be interpreted broadly to include any blockchain-anchored, ledger-registered, cryptographically verifiable digital asset used for representing viewer-provided data, biometric patterns, context variables, wellness states, augmentation interactions, or any transaction generated by system participation. In some embodiments, the tokens may be minted automatically based on algorithmic valuation techniques, transferred across user wallets, traded on internal or external marketplaces, redeemed for access to content layers, or used as governance weights for community-driven augmentation rules.

In some embodiments, the system may represent a unified platform enabling real-time contextual augmentation, VR/AR multi-layered content generation, machine-learning-driven personalization, cross-modal wellness output orchestration, and blockchain-anchored data incentives. In some embodiments, various components may be implemented independently or in combination, and numerous extensions may be envisioned without departing from the spirit of the invention.

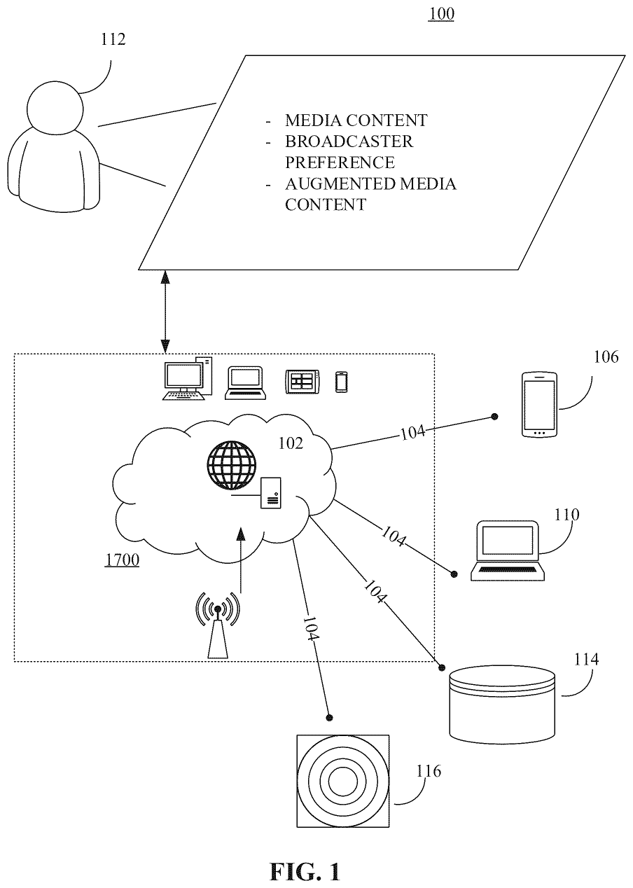

FIG. 1 is an illustration of an online platform 100 consistent with various embodiments of the present disclosure. By way of non-limiting example, the online platform 100 to provide content to viewers for enhancing wellbeing of the viewers may be hosted on a centralized server 102, such as, for example, a cloud computing service. The centralized server 102 may communicate with other network entities, such as, for example, a mobile device 106 (such as a smartphone, a laptop, a tablet computer, etc.), other electronic devices 110 (such as desktop computers, server computers etc.), databases 114, and sensors 116 over a communication network 104, such as, but not limited to, the Internet. Further, users of the online platform 100 may include relevant parties such as, but not limited to, end-users, administrators, service providers, service consumers, and so on. Accordingly, in some instances, electronic devices operated by the one or more relevant parties may be in communication with the platform.

The platform 100 may be embodied as, for example, but not be limited to, a website, a web application, a desktop application, and a mobile application compatible with a computing device. The computing device may comprise, but not be limited to, a desktop computer, laptop, a tablet, or mobile telecommunications device. Moreover, the platform 100 may be hosted on a centralized server, such as, for example, a cloud computing service. Although methods 700 and 800 have been described to be performed by a computing device 1700, it should be understood that, in some embodiments, different operations may be performed by different networked elements in operative communication with computing device 1700.

Embodiments of the present disclosure may comprise a system having a memory storage and a processing unit. The processing unit coupled to the memory storage, wherein the processing unit is configured to perform the stages of methods 700 and 800.

A user 112, such as the one or more relevant parties, may access online platform 100 through a web based software application or browser. The web based software application may be embodied as, for example, but not be limited to, a website, a web application, a desktop application, and a mobile application compatible with a computing device 1700.

Accordingly, in an instance, the user 112, such as a broadcaster may access the platform in order to generate augmented media content. For instance, the broadcaster may provide a media content by either uploading the media content to the platform or providing a hyperlink to the media content. Accordingly, the platform may receive the media content. Further, the broadcaster may provide one or more broadcaster preferences which generally control augmentation of the media content. For example, the broadcaster preferences in general may determine what portion of the media content is to be augmented, which augmentation content is to be selected, how the augmentation is to be performed, to whom the augmented content is to be presented and how the augmented content is to be presented etc. Accordingly, in an instance, the broadcaster may specify one or more context variables (and associated one or more contextual values) based on which the augmentation content may be identified. The one or more contextual variables may be associated with the broadcaster and/or one or more viewers and/or one or more viewer devices. Accordingly, in an instance, the platform may communicate with one or more sensors 116 in order to determine one or more current values corresponding to the one or more contextual variables. Based on a match between the one or more current values with the one or more values specified by the broadcaster, augmentation content may be identified. Further, the online platform may perform content analysis of the media content in order to determine one or more interaction elements (e.g. people, places, brands, etc.). Accordingly, augmentation content identified by the platform may be based on the one or more interaction elements.

Further, in an instance, the augmentation content identified by the platform may be presented to the broadcaster. Accordingly, the broadcaster may provide a confirmation on the augmentation content. Alternatively, in some instances, multiple augmentation content may be presented to the broadcaster and the broadcaster may be enabled to select one or more augmentation content. Further, augmentation content confirmed and/or selected by the broadcaster may then be embedded in the media content to obtain an augmented media content. Such embedding may be performed either based on including the augmentation content per se and/or an indication (e.g. hyperlinks) associated with the augmentation content. In addition, in some instances, the augmented media content may also include the one or more broadcaster preferences. Accordingly, in an instance, when the augmented media content is being played at a viewer device, one or more current values associated with the one or more contextual variables specified in the broadcaster preferences may be determined. Further, a comparison of the one or more current values with the one or more values specified in the broadcaster preferences may be performed. Accordingly, based on a result of the comparison, further filtering of the augmentation content may be performed. As a result, a customized augmented content may be provided to viewers. In addition, in some embodiments, the augmentation content presented to viewers may be based on viewer preferences. Additionally, the online platform may be configured to receive viewer interaction data representing interaction of viewers with the augmented media content. Accordingly, the online platform may control further augmentation of media content based on the viewer interaction data. As a result, the online platform may be configured to discover patterns or trends in viewer behavior and accordingly adapt augmentation of media content.

Although methods 700 and 800 have been described to be performed by platform 100, it should be understood that computing device 1700 may be used to perform the various stages of methods 700 and 800. Furthermore, in some embodiments, different operations may be performed by different networked elements in operative communication with computing device 1700. For example, a server may be employed in the performance of some or all of the stages in methods 700 and 800. Moreover, the server may be configured much like computing device 1700.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of the methods 700 and 800 will be described in greater detail below.

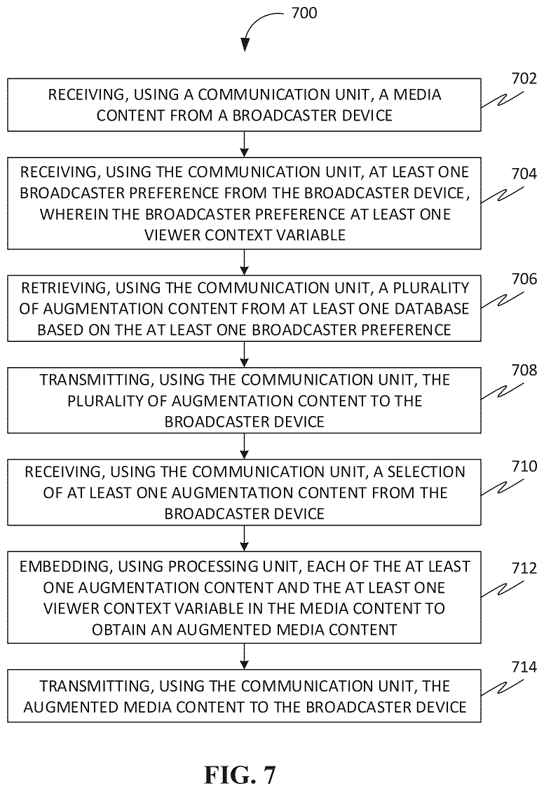

FIG. 7 illustrates a method 700 of providing augmented media content, in accordance with some embodiments. The method 700 may include receiving, using a communication unit, a media content from a broadcaster device. Further, the method 700 may include receiving, using the communication unit, at least one broadcaster preference from the broadcaster device. Further, the broadcaster preference may include at least one viewer context variable. Accordingly, the broadcaster may specify what context variables at a viewer device determine augmentation content. For example, the broadcaster may specify that augmentation content is to be presented based on time, location, sensor data, user characteristics etc. associated with the viewer device.

Further, the method 700 may include a stage 706 of retrieving, using the communication unit, a plurality of augmentation content from at least one database based on the at least one broadcaster preference. Accordingly, in an instance, the at least one database may be associated with third party systems that are in communication with the online platform.

Further, the method 700 may include a stage 708 of transmitting, using the communication unit, the plurality of augmentation content to the broadcaster device. Further, the broadcaster device may be configured to present the plurality of augmentation content. Further, the method 700 may include a stage 710 of receiving, using the communication unit, a selection of at least one augmentation content from the broadcaster device. Accordingly, the plurality of augmentation content may be viewed by a user (i.e. broadcaster) of the broadcaster device prior to making a selection of one or more augmentation content. In some embodiments, the plurality of augmentation content may include links that may enable the broadcaster device to retrieve the plurality of augmentation content from corresponding one or more databases hosting the plurality of augmentation content.

Further, the method 700 may include a stage 712 of embedding, using a processing unit, each of the at least one augmentation content and the at least one viewer context variable in the media content to obtain an augmented media content. Further, presenting of the augmented media content on a viewer device may be based on at least one viewer context value corresponding to the at least one viewer context variable. Further, the at least one viewer context value may be associated with the viewer device. Further, the method 700 may include a stage 714 of transmitting, using the communication unit, the augmented media content to the broadcaster device.

Further, in some embodiments, the augmented media content delivered to the viewer device may subsequently be used by the viewer for further augmenting a part or the whole of the augmented media content as described in conjunction with FIG. 7. Accordingly, in this instance, the viewer device may function as the broadcaster device and the viewer may function as the broadcaster. As a result, users of the platform may successively share augmented media content among themselves that may be generated from one or more executions of media content augmentation as outlined in FIG. 7. In other words, the online platform may facilitate a peer-to-peer exchange of augmented media content between a first user and a second user with one or more augmentations applied prior to each exchange.

In some embodiments, the method 700 may further include receiving, using the communication unit, at least one broadcaster context value corresponding to at least one broadcaster context variable from the broadcaster device. Further, the retrieving of the plurality of augmentation content may be further based on the at least one broadcaster context value. Accordingly, contextual variables of the broadcaster device may also be used to determine relevant augmentation content to be retrieved and embedded into the media content.

In some embodiments, the at least one broadcaster context variable corresponds to at least one broadcaster sensor comprised in the broadcaster device. Further, the at least one viewer context variable corresponds to at least one viewer sensor comprised in the viewer device. Accordingly, in some embodiments, one or more sensors (for sensing motion, orientation, speed etc.) present in the broadcaster device and viewer device may determine the augmentation content to be retrieved/presented.

In some embodiments, the method 700 may further include analyzing, using the processing unit, the media content. Additionally, the method 700 may include identifying, using the processing unit, at least one interaction element in the media content based on the analyzing. Further, the retrieving of the plurality of augmentation content may be further based on the at least one interaction element.

In some embodiments, the method 700 may further include receiving, using the communication unit, viewer interaction data from the viewer device. Further, the viewer interaction data represents interaction of a viewer with the viewer device in association with the augmented content presented on the viewer device. Further, the retrieving of the plurality of augmentation content may be further based on the viewer interaction data.

In some embodiments, the augmented media content may include a plurality of augmented media contents. Accordingly, the viewer interaction data may correspond to a first augmented media content presented at a first time, whereas a second augmented media content may be retrieved based on the viewer interaction data and presented at a second time (later than the first time). Similarly, in some embodiments, the viewer device may include a plurality of viewer devices. Accordingly, interaction data received from a first viewer device may be used to retrieve an augmentation content for presentation on a second viewer device.

In some embodiments, the method 700 may further include receiving, using the communication unit, at least one viewer preference from the viewer device. Further, the retrieving of the plurality of augmentation content may be further based on the at least one viewer preference. Accordingly, viewer preferences may be taken into account by the platform while retrieving relevant augmentation content to be presented to the broadcaster.

In some embodiments, presenting of the augmented media content on the viewer device may be based further on at least one viewer preference. Accordingly, the augmented content may be further filtered at the viewer device end based on one or more viewer preferences.

In some embodiments, the method 700 may further include receiving, using the communication unit, at least one time indicator from the broadcaster device. Further, the at least one time indicator may be associated with play time of the media content. Further, the embedding of the augmentation content may be based on the at least one time indicator. Further, presenting of the augmentation content associated with a time indicator may be synchronous with presenting of the media content corresponding to the time indicator. Accordingly, the broadcaster may specify what points on the time-line of the media content (e.g. video) are to be associated with augmentation content.

In some embodiments, the at least one broadcaster preference may include a conditional statement specifying a condition based on the at least one viewer context variable and an associated augmentation content. Further, the associated augmentation content may be retrieved by the viewer device based on the conditional statement. Accordingly, in addition to augmentation content embedded by the online platform, further augmentation content may be presented to the viewer based on rules specified by the broadcaster. Accordingly, for example, during playtime of a video, augmentation content according to such rules may be retrieved and presented.

In some embodiments, the method 700 may further include receiving, using the communication unit, at least one auto-response content from the broadcaster device. Further, the at least one auto-response content may be associated with the augmentation content. Additionally, the method 700 may include embedding, using the processing unit, the at least one auto-response content in the media content. Further, the at least one auto-response content may be transmitted to at least one communication device associated with the viewer device based on an interaction of a viewer with the viewer device in association with the augmentation content. Accordingly, an SMS/email may be transmitted to the viewer based on an interaction of the viewer with an augmentation content and/or the augmented content. Further, the content of the response may be predetermined by the broadcaster and relevant custom data (e.g. name, contact number etc.) may be included based on characteristics of the viewer device.

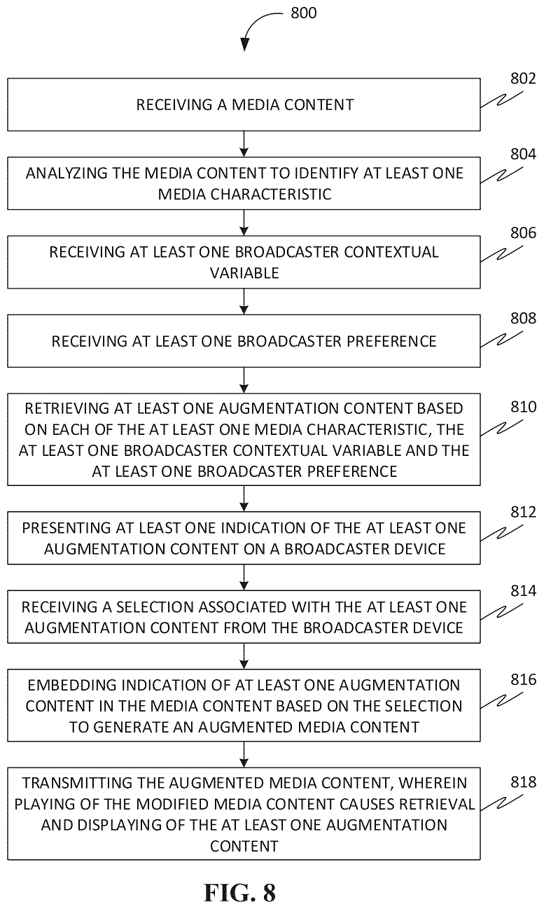

FIG. 8 illustrates a method 800 of providing augmented media content, in accordance with some embodiments. The method 800 may include a stage 802 of receiving a media content from a broadcaster device. Further, the method 800 may include a stage 804 of analyzing the media content to identify at least one media characteristic. Further, the method 800 may include a stage 806 of receiving at least one broadcaster contextual variable. Further, the method 800 may include a stage 808 of receiving at least one broadcaster preference. Further, the method 800 may include a stage 810 of retrieving at least one augmentation content based on each of the at least one media characteristic, the at least one broadcaster contextual variable and the at least one broadcaster preference. Further, the method 800 may include a stage 812 of presenting at least one indication of the at least one augmentation content on a broadcaster device. Further, the method 800 may include a stage 814 of receiving a selection associated with the at least one augmentation content from the broadcaster device. Further, the method 800 may include a stage 816 of embedding indication of at least one augmentation content in the media content based on the selection to generate an augmented media content. Further, the method 800 may include a stage 818 of transmitting the augmented media content, wherein playing of the modified media content causes retrieval and displaying of the at least one augmentation content.

In accordance with some embodiments, a second method of providing augmented media content may be provided. The second method may include receiving, using the processing unit, at least one broadcaster preference. Further, the second method may include receiving, using a processing unit, a media content from a media source. Further, the second method may include analyzing, using the processing unit, the media content. Further, the second method may include identifying, using the processing unit, at least one interaction element in the media content based on the analyzing. Further, the second method may include identifying, using the processing unit, at least one augmentation content based on each of the at least one interaction element and the at least one broadcaster preference. Further, the second method may include embedding, using the processing unit, the at least one augmentation content in the media content to obtain an augmented media content. Further, the second method may include transmitting, using a communication unit, the augmented media content to a viewer device. Further, the viewer device may be configured to present the media content. Further, the viewer device may be associated with at least one viewer preference and at least one viewer contextual data. Further, presenting of the at least one augmentation content may be based on the at least one viewer preference and the at least one viewer contextual data.

In accordance with some embodiments, a third method of providing augmented media content is provided. The third method may include receiving, using a communication unit, a media content from a broadcaster device. Further, the third method may include receiving, using the communication unit, at least one broadcaster contextual data from the broadcaster device. Further, the at least one broadcaster contextual data may be associated with one or more of the media content and a broadcaster associated with the user device. Further, the third method may include analyzing, using a processing unit, one or more of the media content and the at least one broadcaster contextual data. Further, the third method may include identifying, using the processing unit, a plurality of augmentation content based on the analyzing. Further, the third method may include transmitting, using the communication unit, the plurality of augmentation content to the broadcaster device. Further, the third method may include receiving, using the communication unit, a selection of at least one augmentation content from the plurality of augmentation content. Further, the third method may include embedding, using the processing, one or more of the at least one augmentation content in the media content to obtain an augmented media content. Further, the third method may include transmitting, using the communication unit, the augmented media content to the broadcaster device.

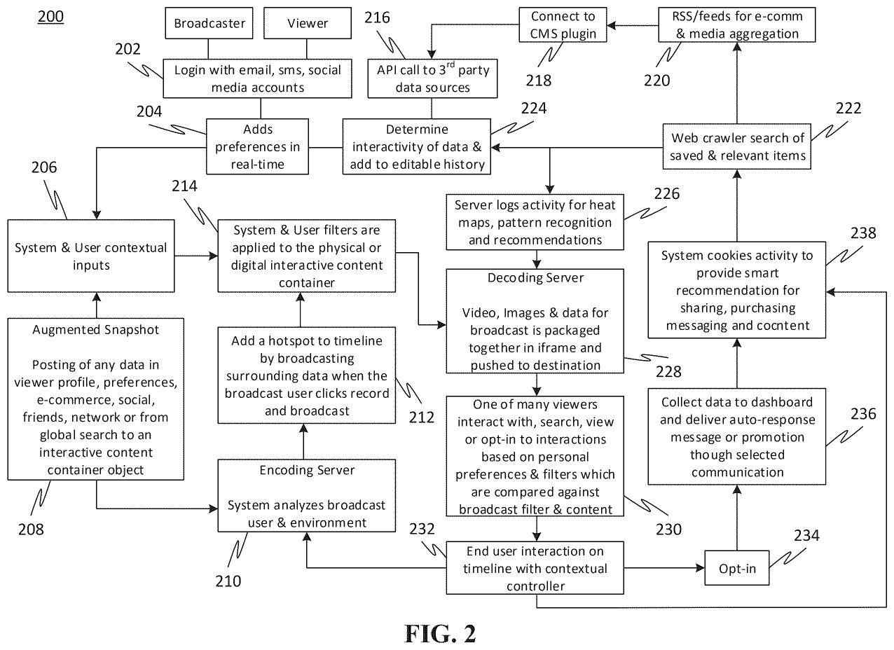

FIG. 2 illustrates a flowchart of a method 200 of generating an augmented media content, in accordance with some embodiments. At step 202, a broadcaster and/or a viewer may login with email, SMS, social media accounts etc. At step 204, the broadcaster and/or the viewer may add preferences in real-time. At step 206, System and User contextual inputs may be received from the broadcaster and/or the viewer. In an instance, such inputs and contextual data may include environmental data, historical data, physical movements (of eyes, body etc.), facial and pattern recognition, image recognition, user preferences, IOT/WiFi/BLE/RF, social media, friends, trends data, GPS data, time, tags, advertisements, promotions, 3rd party videos etc. Further, at step 208, posting of any data in viewer profile, preferences, e-commerce, social, friends, network or from global search may be added to an interactive content container object (i.e. Augmented Snapshot). At step 210, broadcast user and environment may be analyzed. This may include for example, direction of all cameras, distance, location, elevation, position, directional movement, eye movement, mouse or gesture movement, environmental conditions, trends etc. Further, at step 212, the broadcaster may add a hotspot to timeline by broadcasting surrounding data when the broadcaster clicks record and broadcast. Further, at step 214, System and User filters may be applied to the physical or digital interactive content container. This may include, e-commerce, location/time/speed, social media, people, media content, searchable web, IOT, ad-networks etc. In order to retrieve augmentation content, a step 216 may be executed. Accordingly, API calls to 3rd party data sources may be invoked. Additionally, at step 218, connection to a CMS plugin may be established in order to obtain the augmentation content. Further, at step 220, RSS/feeds for e-commerce and media aggregation may be performed. Further, at step 222, Web crawler search of saved and relevant items may be performed. Additionally, at step 224, interactivity of data may be determined and added to editable history. Further, activity for heat maps, pattern recognition and recommendations may be logged. Further, at step 228, Video, Images and data for broadcast may be packaged together in an iframe and pushed to destination (i.e. a viewer device). Alternatively, the augmented content may be directly shared or sent to viewer via web, Bluetooth, SMS or other data transfer method. Further, in another instance, the augmented content may be broadcasted to one or more many embedded iframes on multiple destinations. Further, the viewing filters may be created based on the System and User contextual inputs available. Further, at step 230, one of many viewers may interact with, search, view or opt-in to interactions based on personal preferences and filters which are compared against broadcaster filter and content. Further, at step 232, end user interaction on timeline with contextual controller may be performed. This may include media, text, social media, email etc. Further, at step 234, viewers may opt-in for providing interaction data and/or receiving promotions, deals etc. Accordingly, at step 236, interaction data may be collected and presented on the dashboard and auto-response message or promotion may be delivered to viewers though selected communication (e.g. SMS, email, IM, social media etc.). Further, at step 238, cookies may track activity to provide smart recommendation for sharing, purchasing messaging and content.

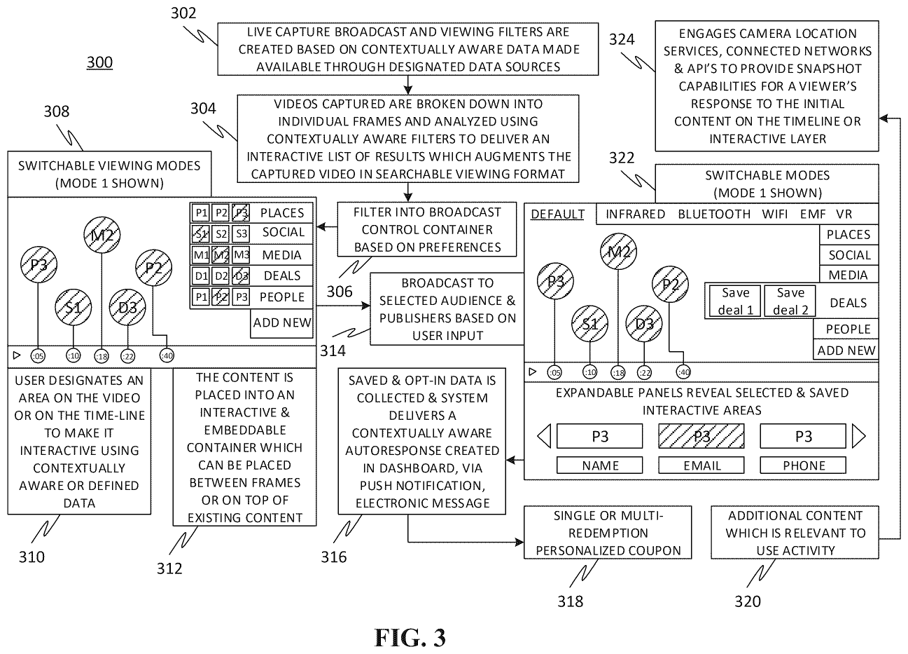

FIG. 3 illustrates a flowchart of a method 300 of generating an augmented media content including a depiction of user interfaces presented on a broadcaster device and a viewer device, in accordance with some embodiments. At step 302, live capture, broadcast and viewing filters are created based on contextually aware data made available through designated data sources. At step 304, videos captured are broken down into individual frames and analyzed using contextually aware filters to deliver an interactive list of results which augments the captured video in a searchable viewing format. At step 306, filtering into broadcast control container based on preferences may be performed. At step 308, a switchable viewing mode may be selected. Further, at step 310, the user (e.g. a broadcaster) designates an area on the video or on the time-line to make it interactive using contextually aware or defined data. For example, as illustrated, the broadcaster may identify one or more time points on the time-line of the video where augmented content is to be include. Further, for each time-point selected by the broadcaster, multiple augmented content may be presented for selection. For example, as shown, a plurality of categories of augmented content may presented, such as, but not limited to, places (P1, P2, P3), social (S1, S2, S3), media (M1, M2, M3), deals (D1, D2, D3), people (P1, P2, P3). Accordingly, the broadcaster may select one or more instances of one or more categories. For example, as shown, the broadcaster's selection of P3, S1, M2, D3, P2 corresponding to respective plurality of categories may be performed. Further, in some instances, the broadcaster may be enabled to include a new category.

Accordingly, at step 312, the content is placed into an interactive and embeddable container which can be placed between frames or on top of existing content. Further, at step 314, the content with the interactive and embeddable container may be broadcast to selected audience and publishers based on user input. At step 316, saved and opt-in data may be collected and system may deliver a contextually aware auto-response created in dashboard, via push notification, electronic messaging (e.g. SMS, email, Chat, social media, etc.). Further, at step 318 single or multi-redemption personalized coupon may be delivered. Further, at step 320, additional content which is relevant to user activity may also be delivered. Accordingly, at step 322, a viewer may select one or a plurality of switchable viewing modes and accordingly view the delivered content. Further, at step 324, engagement with camera, location, services, connected networks and API's may be performed to provide snapshot capabilities for a viewer's response to the initial content on the timeline or interactive layer.

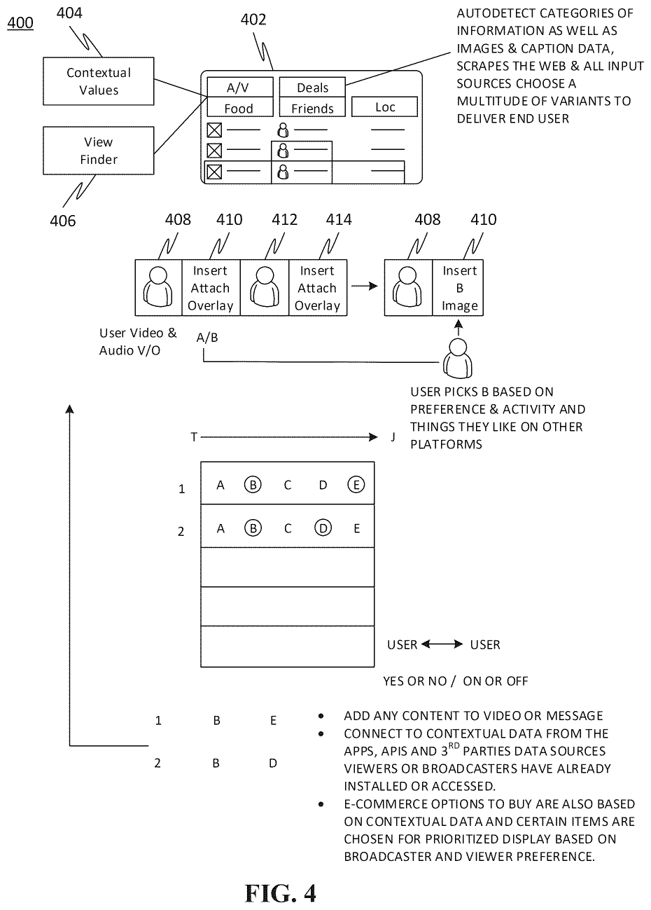

FIG. 4 illustrates embedding of augmentation content in a media content for generating an augmented media content, in accordance with some embodiments. As shown, the media content may be analyzed and a plurality of categories of augmentation content may be identified based on the analysis and presented to the broadcaster in user interface 402 on the broadcaster device. Further, the augmentation content may be identified based on contextual values 404 that may be determined using, for example, sensors associated with the broadcaster device and/or the viewer device. Additionally, the augmentation content may be identified based on view finder status. Accordingly, the augmentation content may be relevant only to a portion of media content associated with the view finder status. The view finder status may correspond to one or more of the broadcaster device and the viewer device. Further illustrated is the embedding of the augmentation content into the media content. In an instance, the augmentation content may be embedded in between the frames of media content. For example, augmentation content may be embedded in slots 410 and 414 interspersing media content frames 408 and 414. In an instance, the augmentation content embedded in slot 410 may include images corresponding to users A and B. Accordingly, when the user B receives the augmented media content, the image B is retrieved and presented to the user B based on preferences and/or activity of user B.

Likewise, a plurality of augmentation content corresponding to multiple users A, B, C, D and E may be embedded in the media content. Accordingly, when the augmented media content is presented on a viewer device, a respective augmentation content may be retrieved and presented to the user of the viewer device. Further, each user may specify multiple modes, wherein each mode may correspond to a filtering of the augmentation content.

Accordingly, the platform may allow adding any content to a video or message. Subsequently, the platform facilitates connecting to contextual data from apps, APIs and 3rd party data sources already installed or accessed by viewers or broadcasters. Further, the platform also enables E-Commerce options to buy based on contextual data and certain items are chosen for prioritized display based on broadcaster and viewer preferences.

Further, in some embodiments, the viewer can respond with their own augmented content back to the broadcaster or share via 2-way multimedia messaging and interaction. Accordingly, the platform in some embodiments may function as a social media platform while enabling users to augment contextually aware content in media items, such as, but not limited to, videos.

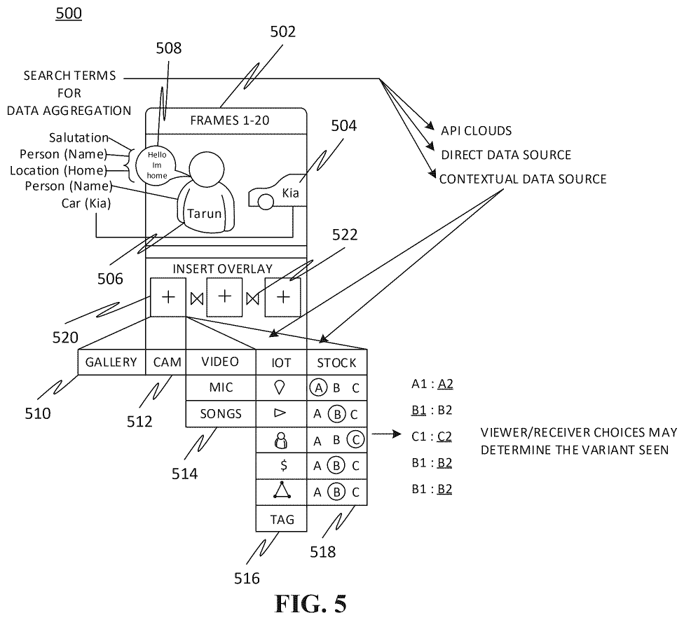

FIG. 5 illustrates an exemplary user interface 500 presentable on a broadcaster device for facilitating augmentation of media content, in accordance with some embodiments. In an instance, the media content may be analyzed to detect interaction points (e.g. people, places, speech, products, brands, etc.). As illustrated, analysis of frames 1-20 of media content 502 may indicate presence of product 504 (e.g. car KIA), person 506 and message 508 (e.g. greeting). Such analysis may accordingly include, speech recognition, face detection, object detection and so on. Accordingly, based on the detected interaction points, a plurality of augmentation content may be identified and presented to the broadcaster. Further, the analysis of the media content and/or retrieval of augmentation content may be performed by accessing API clouds, direct data sources and contextual data sources. Further, in some embodiments, the detected interaction points may be presented to the broadcaster and a feedback (e.g. confirmation or corrections) may be received. As a result, accuracy of interaction points detection may be improved. Additionally, the interaction points detected may be used for searching the media content and/or the augmented media content based on search terms identified as a result of detecting the interaction points.

In an instance, the user interface may include an “Insert Overlay” GUI element showing two options (insert or overlay) which can be applied to any uploaded clip or contextual content. The GUI elements 520 (boxes with the plus signs) allow the broadcaster to add media content and/or augmentation content, while GUI elements 522 (triangle shaped) in between are for transition options between content.

Further, the plurality of augmentation content may be provided in the form of predetermined categories 516 (e.g. IOT, location, media, person, e-commerce/deals, social/sharing, tags, etc. Further, the augmentation content may include stock images 518 that may be selected and presented to viewers based on contextual data and/or viewer preferences. In an instance, the stock images 518 may be present on a local storage associated with the broadcaster device and/or the viewer device.

Further, the user interface may enable a user, such as the broadcaster, to select the media content from one or more sources (e.g. gallery 510, camera 512, media source 514 that may include video, microphone and songs). Further, the broadcaster can add content via Mic or Camera input (viewfinder, photo/video/audio) or uploaded content source (e.g. Youtube™ or library). Accordingly, all uploaded or real-time (frame based or still image recognition in viewfinder) content can be scanned by image/facial recognition and parsed against databases of content or used as queries to contextual data sources.

Additionally, the user interface may enable a viewer to choose yes/no or toggle contextual suggestions/survey and variants from the broadcaster which conditionally prompt an action.

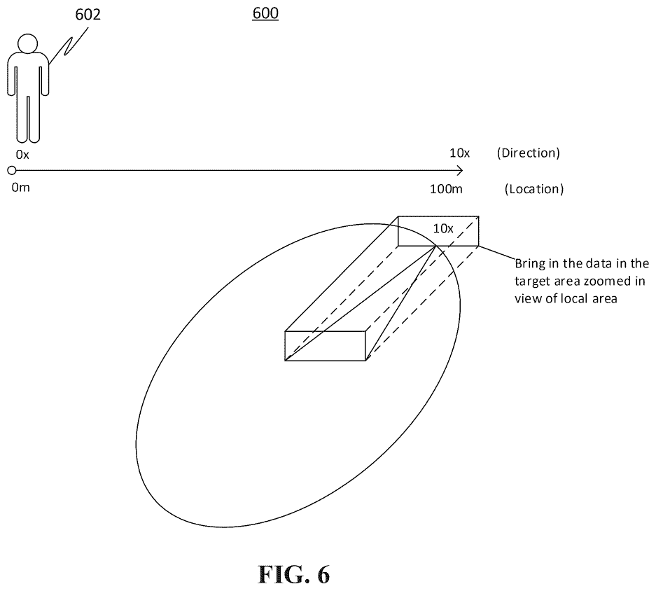

FIG. 6 illustrates augmentation of media content based on viewing region (as characterized by zoom factor, distance etc.) in accordance with some embodiments. Accordingly, a dynamic zoom of the augmented media content may be provided. For instance, if a radius of a user 602 is 10 m or 1× zoom, the user 602 may see contextual data or augmented reality within that area. If the user 602 chooses 100 m or 10× zoom, the viewfinder may display related content around that area for augmented reality and contextual broadcasting. More generally, in some embodiments, the augmentation content presented to a viewer may be based on a portion of the media content being viewed/consumed by the viewer. The portion may correspond to a particular region in space, or an interval in time or both. Accordingly, in some embodiments, the portion of the media content being currently consumed by the viewer may be determined based on view finder status, gaze tracking, and so on. As a result, in some instances, augmentation content that is relevant to a current viewing/listening context may be identified and presented.

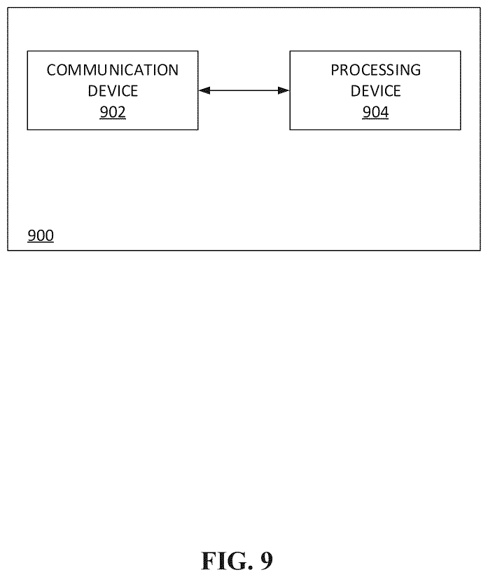

FIG. 9 is a block diagram of a system 900 for providing content to viewers for enhancing wellbeing of the viewers, in accordance with some embodiments. Accordingly, the system 900 may include a communication device 902 and a processing device 904. Further, the communication device 902 may include a communication unit. Further, the processing device 904 may include a processing unit.

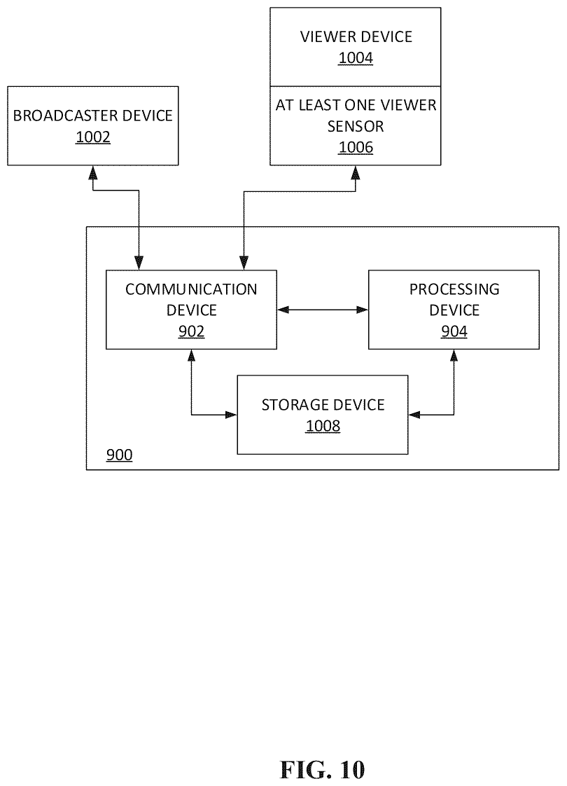

Further, the communication device 902 may be configured for receiving a media content from a broadcaster device 1002, as shown in FIG. 10. Further, the broadcaster device 1002 may be associated with a broadcaster. Further, the broadcaster may be an artificial intelligence (AI) entity. Further, the media content may include a plurality of content from the broadcaster device 1002. Further, the plurality of content may include a plurality of signals from a plurality of input devices comprised in the broadcaster device 1002. Further, the plurality of signals may include a plurality of analog input signals and a plurality of digital input signals. Further, the plurality of analog input signals and the plurality of digital input signals may include light signals, sound signals, vibration signals, haptic signals, biometric signals, scalar signals, radio signals, TV signals, Zigbee signals, WiFi signals, LiFi signals, IR signals, Bluetooth signals, projections, VR (virtual reality) content, etc. Further, the plurality of devices may include a light device, a sound device, a vibration device, a haptic device, a biometric device, a scalar device, a radio, a TV, a Zigbee device, a WiFi device, a LiFi device, a BLE device, an IR device, a projector, a VR device, a pro audio device, a mobile device, a desktop device, etc. Further, the broadcaster device 1002 may include a computing device, a client device, etc. Further, in an instance, the plurality of signals from the plurality of input devices may be used to generate a geometry or render an object in a virtual reality environment (such as Metaverse™). Further, the geometry and the object may be used as a NFT (Non-Fungible Token). Further, the communication device 902 may be configured for receiving at least one broadcaster preference from the broadcaster device 1002. Further, the at least one broadcaster preference may include at least one viewer context variable. Further, the at least one viewer context variable may correspond to user data, trends, historical preferences, and existing databases associated with the viewers. Further, the communication device 902 may be configured for transmitting an augmented media content to a viewer device 1004, as shown in FIG. 10. Further, the augmented media content may include an audio content, a video content, an audio-video content, a haptic content, a virtual reality (VR) content, an augmented reality (AR) content, etc. Further, the viewer device 1004 may include a computing device, a client device, an output device, an input device, etc. Further, the viewer device 1004 may be configured for presenting the augmented media content to a viewer. Further, the viewer device 1004 may include a receiver device associated with a receiver. Further, the viewer may include the receiver. Further, the viewer may include a user, an individual, etc. Further, the presenting of the augmented media content on the viewer device 1004 may be based on at least one viewer context value corresponding to the at least one viewer context variable. Further, the viewer device 1004 may include a plurality of analog devices and a plurality of digital output devices. Further, the plurality of analog devices and the plurality of digital output devices may include a light device, a sound device, a vibration device, a haptic device, a biofeedback device, a scalar device, a radio, a TV, a binaural device, a multi-phase device, a Zigbee device, a WiFi device, a LiFi device, a BLE device, an IR device, a projection device, an IOT device, a Virtual Assistant device, a VR device, a mobile device, and a desktop device in home or in air/road travel environments. Further, the at least one viewer context variable corresponds to at least one viewer sensor 1006 (one or more sensors), as shown in FIG. 10, comprised in the viewer device 1004. Further, the at least one viewer sensor 1006 may be a human implantable device, an implantable sensor, etc. Further, the at least one viewer sensor 1006 may include a camera, a microphone, a gesture sensor, a motion sensor, a biological sensor, a physiological sensor, a physical sensor, a biokinetic sensor, etc. Further, the at least one viewer sensor 1006 may be configured for generating the at least one viewer context value for the at least one viewer context variable based on detecting a response of the viewer corresponding to the augmented media content. Further, in an instance, the detecting of the response of the viewer may include detecting the response of the viewer in real time. Further, the response may include a voluntary response, an involuntary response, etc. of the viewer. Further, the communication device 902 may be configured for transmitting the augmented media content to the broadcaster device 1002. Further, the at least one viewer context variable may include a classification of the response. Further, the at least one viewer context value may include a level of the response provided by the viewer for the classification of the response.

Further, the processing device 904 may be communicatively coupled with the communication device 902. Further, the processing device 904 may be configured for generating the augmented media content for the enhancing of the wellbeing of the viewer based on the at least one viewer context variable and the media content. Further, the augmented media content may include a layered content. Further, the layered content may include layers of audio, visual, haptic, XR, consumable content, etc. Further, the generating of the augmented media content may include at least one of an individual and a combinational placing or augmenting of the layers of the audio, the visual, the haptic, the XR, the consumable content, etc. onto a base layer of a determined size, a layout, a format, a color, a frequency, a vibration, a music key, a tone, a beat per minute, a displacement, an orientation, a phase, an amplitude, a pulse width, a modulation, a prilling, a cymatic, a sequence, a geometry, a network connection, and any relevant output. Further, the generating of the augmented media content may include generating the augmented media content using at least one machine learning model. Further, the at least one machine learning model may include at least one generative algorithm. Further, the augmented media content comprises the media content and the at least one viewer context variable. Further, the enhancing of the wellbeing of the viewer may include providing entertainment, therapy, treatment, etc. to the viewer.

Further, in some embodiments, the detecting of the response may include detecting a change in at least one of a physical state, a psychological state, and a biological state of the viewer. Further, the physical state corresponds to a body temperature of the viewer. Further, the physiological state corresponds to a heart rate, a respiration rate, a pulse rate, a blood pressure, etc. of the viewer. Further, the biological state corresponds to a level of one or more hormones, cellular or genetic composition in a body of the viewer. Further, the generating of the at least one viewer context value for the at least one viewer context variable may be based on the detecting of the change in at least one of the physical state, the psychological state, and the biological state of the viewer over a fixed or adaptive period of time and as it relates to historical, comparative and relational data in context.

Further, in some embodiments, the detecting of the response may include detecting an expression of the viewer corresponding to the augmented media content. Further, the expression may include a verbal expression, a facial expression, a gesture, etc. Further, the generating of the at least one viewer context value for the at least one viewer context variable may be based on the detecting of the expression of the viewer corresponding to the augmented media content.

In further embodiments, the system 900 may include a storage device 1008, as shown in FIG. 10. Further, the storage device 1008 may include a memory storage. Further, the storage device 1008 may be communicatively coupled with the communication device 902 and the processing device 904. Further, the storage device 1008 may be configured for retrieving a plurality of augmentation content from at least one database based on the at least one broadcaster preference. Further, the communication device 902 may be configured for transmitting the plurality of augmentation content to the broadcaster device. Further, the broadcaster device may be configured to present the plurality of augmentation content. Further, the communication device 902 may be configured for receiving a selection of at least one augmentation content from the broadcaster device. Further, the generating of the augmented media content may include embedding each of the at least one augmentation content and the at least one viewer context variable in the media content to obtain the augmented media content.

Further, in an embodiment, the communication device 902 may be configured for receiving the at least one viewer context value corresponding to the at least one viewer context variable from the viewer device 1004. Further, the processing device 904 may be configured for analyzing the at least one viewer context value and the at least one viewer context variable corresponding to the at least one viewer context value using at least one second machine learning model. Further, the at least one second machine learning model may be contextually aware. Further, the processing device 904 may be configured for generating a health data for the viewer based on the analyzing of the at least one viewer context value. Further, the health data may include a health profile of the viewer. Further, the storage device 1008 may be configured for storing the health data of the viewer in a distributed ledger.

Further, in some embodiments, the communication device 902 may be configured for transmitting a transactional request for acquiring the health data of the viewer to the viewer device 1004. Further, the communication device 902 may be configured for receiving a first response for the transactional request from the viewer device 1004. Further, the first response may include a confirmation of the acquisition of the health data. Further, the communication device 902 may be configured for transmitting a number of cryptocurrency tokens to the viewer device 1004. Further, the storing of the health data may be based on the transmitting of the number of cryptocurrency tokens. Further, the processing device 904 may be configured for analyzing the health data of the viewer based on the first response. Further, the processing device 904 may be configured for determining a value of the health data of the viewer based on the analyzing of the health data. Further, the processing device 904 may be configured for generating the number of cryptocurrency tokens for the health data based on the value of the health data. Further, the number of cryptocurrency tokens may be monetized. Further, the number of cryptocurrency tokens may be associated with a monetary value. Further, the number of cryptocurrency tokens may be tradable.

Further, in some embodiments, the generating of the number of cryptocurrency tokens may include minting the number of cryptocurrency tokens for the health data based on the value of the health data.

Further, in some embodiments, the storage device 1008 may be configured for retrieving a plurality of health data of a plurality of viewers. Further, the storage device 1008 may be configured for storing the plurality of augmentation content in the at least one database. Further, the processing device 904 may be configured for analyzing the plurality of health data. Further, the processing device 904 may be configured for generating the plurality of augmentation content based on the analyzing of the plurality of health data.

Further, in some embodiments, the analyzing of the plurality of health data may include analyzing the plurality of health data using at least one machine learning model. Further, the at least one machine learning model may be contextually aware. Further, the generating of the plurality of augmentation content may be based on the analyzing of the plurality of health data using the at least one machine learning model.

Further, in some embodiments, the communication device 902 may be configured for receiving at least one viewer data associated with the viewer from the viewer device 1004. Further, the at least one viewer data may include personal information, preferences, etc. Further, the processing device 904 may be configured for analyzing the at least one viewer data using at least one first machine learning model. Further, the at least one first machine learning model may be contextually aware. Further, the processing device 904 may be configured for determining at least one requirement of the viewer for the enhancing of the wellbeing of the viewer and the at least one viewer context variable corresponding to the at least one requirement based on the analyzing of the at least one viewer data. Further, the at least one requirement may include a particular genre of entertainment, a particular therapy, etc. Further, the processing device 904 may be configured for identifying a selection of at least one first augmentation content from the plurality of augmentation content based on the at least one requirement. Further, the processing device 904 may be configured for embedding each of the at least one first augmentation content and the at least one viewer context variable in the media content to further obtain the augmented media content for the enhancing of the wellbeing of the viewer.

FIG. 10 is a block diagram of the system 900 for providing the content to the viewers for enhancing wellbeing of the viewers, in accordance with some embodiments.

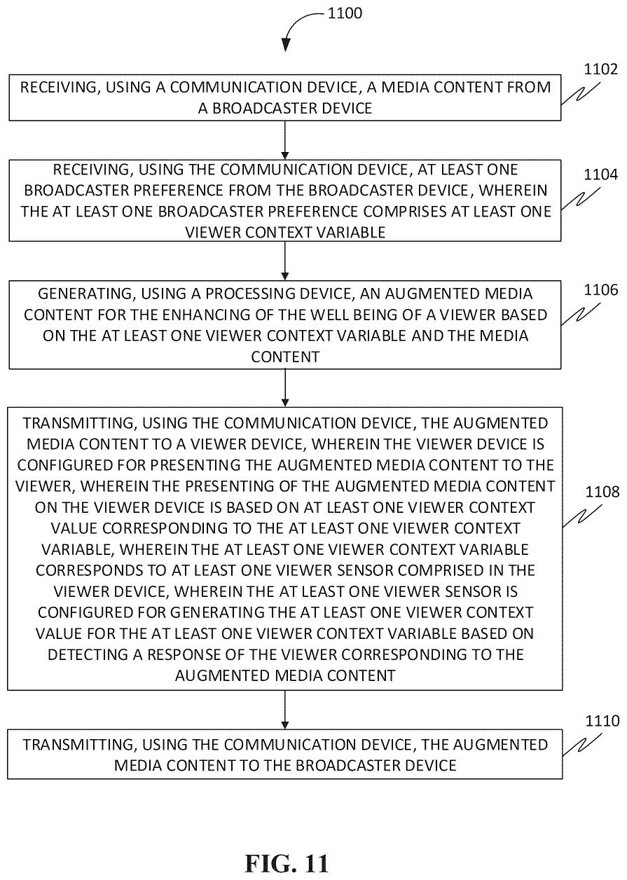

FIG. 11 is a flowchart of a method 1100 for providing content to viewers for enhancing wellbeing of the viewers, in accordance with some embodiments. Accordingly, at 1102, the method 1100 may include receiving, using a communication device, a media content from a broadcaster device.

Further, at 1104, the method 1100 may include receiving, using the communication device, at least one broadcaster preference from the broadcaster device. Further, the at least one broadcaster preference may include at least one viewer context variable.

Further, at 1106, the method 1100 may include generating, using a processing device, an augmented media content for the enhancing of the wellbeing of a viewer based on the at least one viewer context variable and the media content. Further, the augmented media content may include the media content and the at least one viewer context variable.

Further, at 1108, the method 1100 may include transmitting, using the communication device, the augmented media content to a viewer device. Further, the viewer device may be configured for presenting the augmented media content to the viewer. Further, the presenting of the augmented media content on the viewer device may be based on at least one viewer context value corresponding to the at least one viewer context variable. Further, the at least one viewer context variable corresponds to at least one viewer sensor comprised in the viewer device. Further, the at least one viewer sensor may be configured for generating the at least one viewer context value for the at least one viewer context variable based on detecting a response of the viewer corresponding to the augmented media content.

Further, at 1110, the method 1100 may include transmitting, using the communication device, the augmented media content to the broadcaster device.

Further, in some embodiments, the detecting of the response may include detecting a change in at least one of a physical state, a psychological state, and a biological state of the viewer. Further, the generating of the at least one viewer context value for the at least one viewer context variable may be based on the detecting of the change in at least one of the physical state, the psychological state, and the biological state of the viewer.

Further, in some embodiments, the detecting of the response may include detecting an expression of the viewer corresponding to the augmented media content. Further, the generating of the at least one viewer context value for the at least one viewer context variable may be based on the detecting of the expression of the viewer corresponding to the augmented media content.

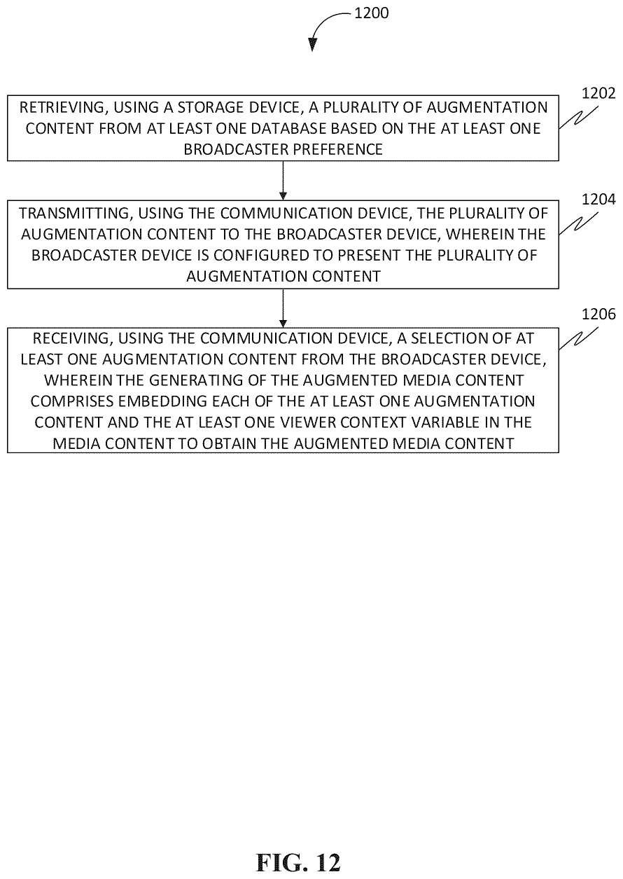

FIG. 12 is a flowchart of a method 1200 for embedding augmentation content in the media content for providing the content to the viewers for enhancing the wellbeing of the viewers, in accordance with some embodiments. Further, at 1202, the method 1200 may include retrieving, using a storage device, a plurality of augmentation content from at least one database based on the at least one broadcaster preference.

Further, at 1204, the method 1200 may include transmitting, using the communication device, the plurality of augmentation content to the broadcaster device. Further, the broadcaster device may be configured to present the plurality of augmentation content.

Further, at 1206, the method 1200 may include receiving, using the communication device, a selection of at least one augmentation content from the broadcaster device. Further, the generating of the augmented media content may include embedding each of the at least one augmentation content and the at least one viewer context variable in the media content to obtain the augmented media content.

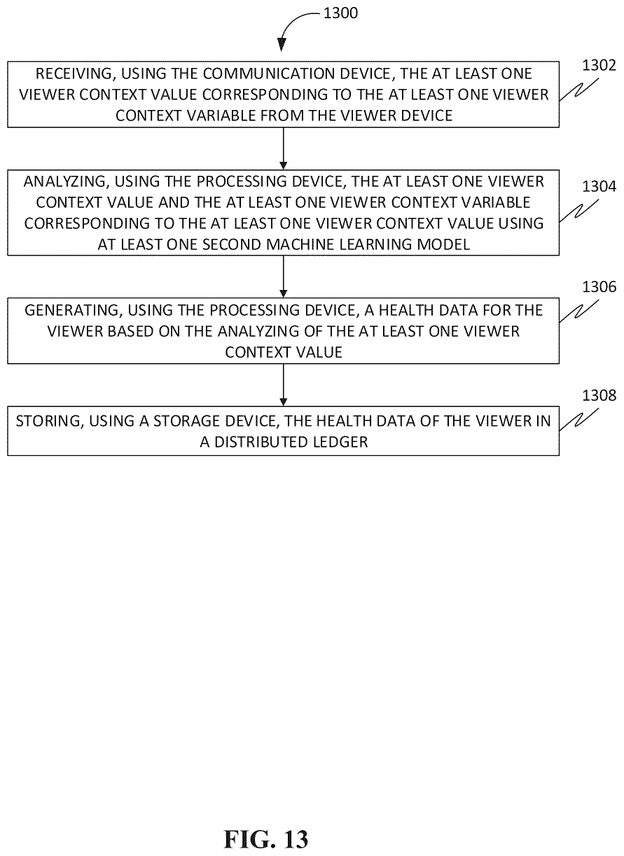

FIG. 13 is a flowchart of a method 1300 for generating health data for the viewers for providing the content to the viewers, in accordance with some embodiments. Accordingly, at 1302, the method 1300 may include receiving, using the communication device, the at least one viewer context value corresponding to the at least one viewer context variable from the viewer device.

Further, at 1304, the method 1300 may include analyzing, using the processing device, the at least one viewer context value and the at least one viewer context variable corresponding to the at least one viewer context value using at least one second machine learning model.

Further, at 1306, the method 1300 may include generating, using the processing device, a health data for the viewer based on the analyzing of the at least one viewer context value.

Further, at 1308, the method 1300 may include storing, using the storage device, the health data of the viewer in a distributed ledger.

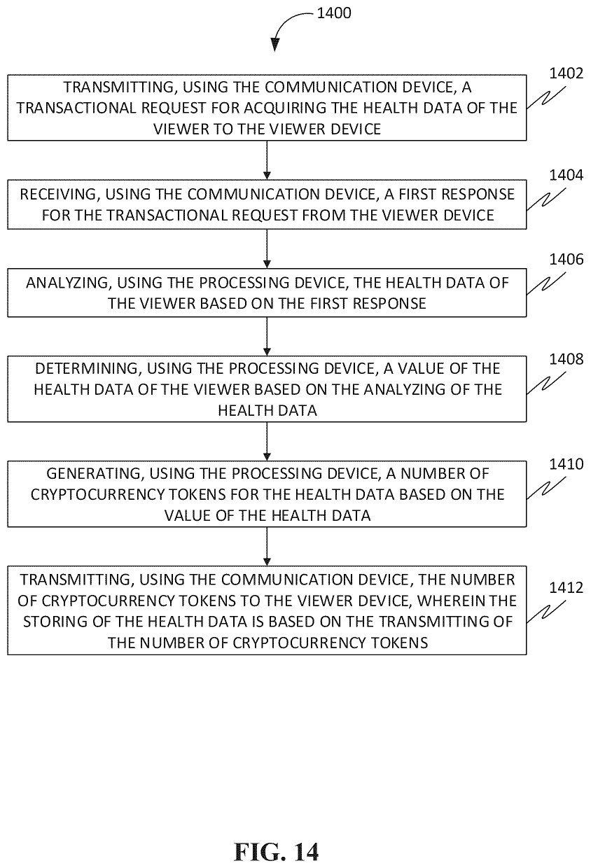

FIG. 14 is a flowchart of a method 1400 for acquiring the health data from the viewers for providing the content to the viewers, in accordance with some embodiments. Accordingly, at 1402, the method 1400 may include transmitting, using the communication device, a transactional request for acquiring the health data of the viewer to the viewer device.

Further, at 1404, the method 1400 may include receiving, using the communication device, a first response for the transactional request from the viewer device.

Further, at 1406, the method 1400 may include analyzing, using the processing device, the health data of the viewer based on the first response.

Further, at 1408, the method 1400 may include determining, using the processing device, a value of the health data of the viewer based on the analyzing of the health data.

Further, at 1410, the method 1400 may include generating, using the processing device, a number of cryptocurrency tokens for the health data based on the value of the health data.

Further, at 1412, the method 1400 may include transmitting, using the communication device, the number of cryptocurrency tokens to the viewer device. Further, the storing of the health data may be based on the transmitting of the number of cryptocurrency tokens.

Further, in some embodiments, the generating of the number of cryptocurrency tokens may include minting the number of cryptocurrency tokens for the health data based on the value of the health data.

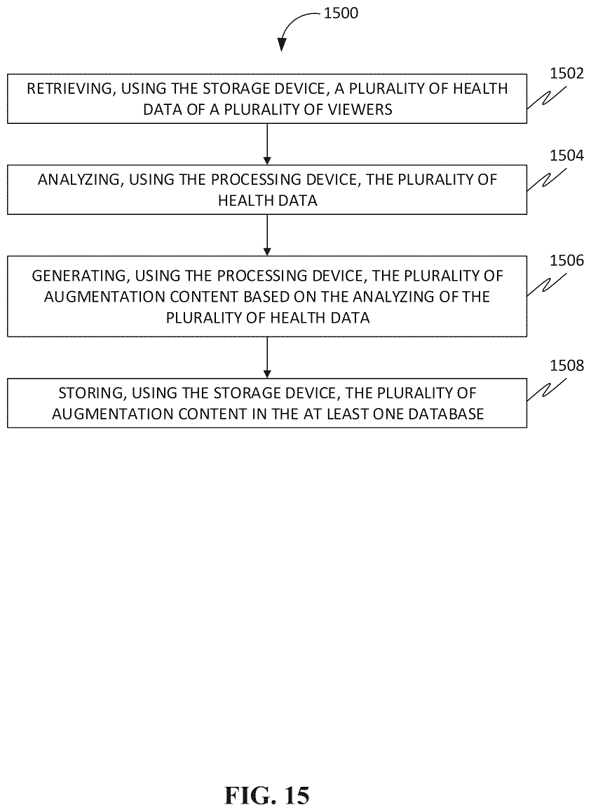

FIG. 15 is a flowchart of a method 1500 for generating augmentation content for providing the content to the viewers, in accordance with some embodiments.

Accordingly, at 1502, the method 1500 may include retrieving, using the storage device, a plurality of health data of a plurality of viewers.

Further, at 1504, the method 1500 may include analyzing, using the processing device, the plurality of health data.

Further, at 1506, the method 1500 may include generating, using the processing device, the plurality of augmentation content based on the analyzing of the plurality of health data.

Further, at 1508, the method 1500 may include storing, using the storage device, the plurality of augmentation content in the at least one database.

Further, in some embodiments, the analyzing of the plurality of health data may include analyzing the plurality of health data using at least one machine learning model. Further, the generating of the plurality of augmentation content may be based on the analyzing of the plurality of health data using the at least one machine learning model.

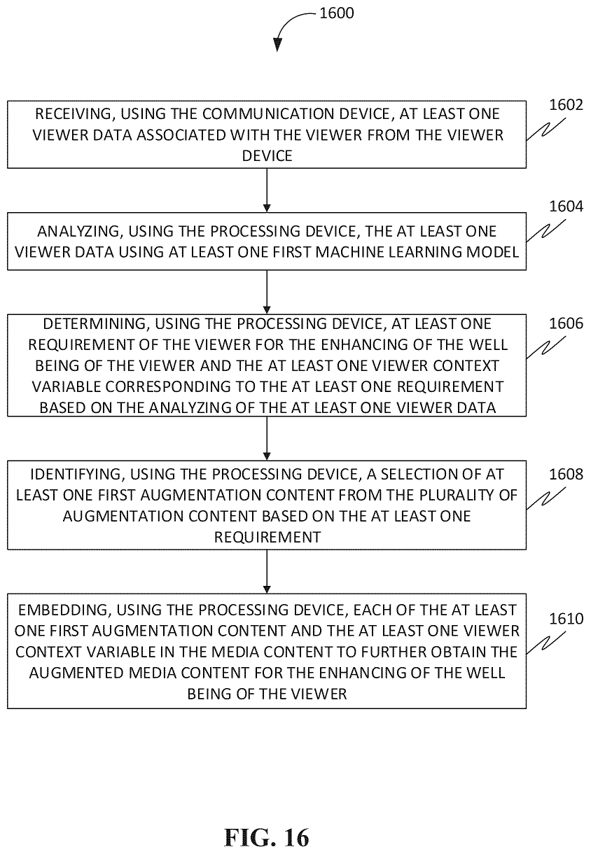

FIG. 16 is a flowchart of a method 1600 for determining requirements of the viewers for providing the content to the viewers, in accordance with some embodiments. Accordingly, at 1602, the method 1600 may include receiving, using the communication device, at least one viewer data associated with the viewer from the viewer device.

Further, at 1604, the method 1600 may include analyzing, using the processing device, the at least one viewer data using at least one first machine learning model analyzing, using the processing device, the at least one viewer data using at least one first machine learning model.

Further, at 1606, the method 1600 may include determining, using the processing device, at least one requirement of the viewer for the enhancing of the wellbeing of the viewer and the at least one viewer context variable corresponding to the at least one requirement based on the analyzing of the at least one viewer data.

Further, at 1608, the method 1600 may include identifying, using the processing device, a selection of at least one first augmentation content from the plurality of augmentation content based on the at least one requirement.

Further, at 1610, the method 1600 may include embedding, using the processing device, each of the at least one first augmentation content and the at least one viewer context variable in the media content to further obtain the augmented media content for the enhancing of the wellbeing of the viewer.

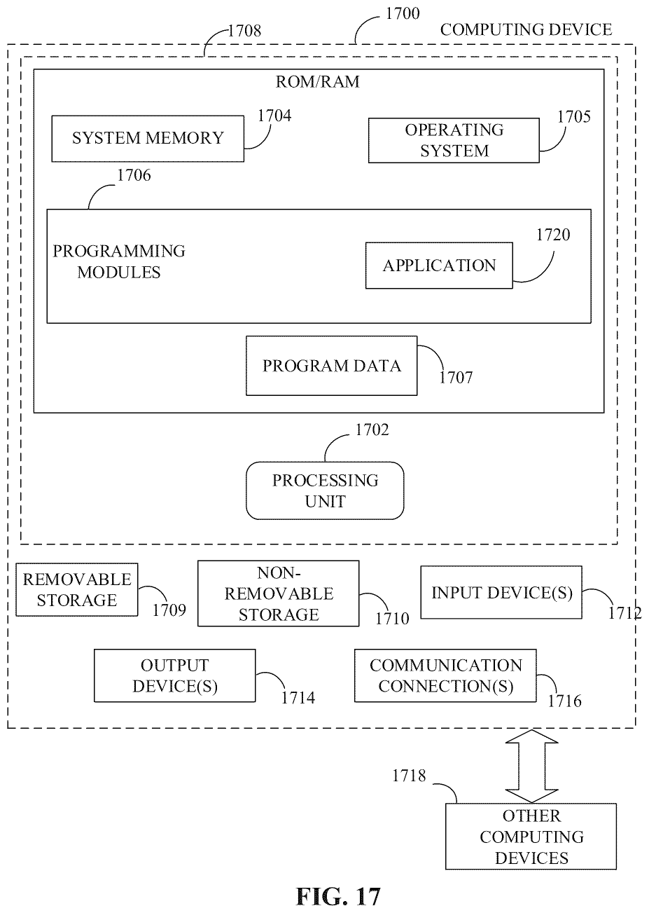

With reference to FIG. 17, a system consistent with an embodiment of the disclosure may include a computing device or cloud service, such as computing device 1700. In a basic configuration, computing device 1700 may include at least one processing unit 1702 and a system memory 1704. Depending on the configuration and type of computing device, system memory 1704 may comprise, but is not limited to, volatile (e.g. random-access memory (RAM)), non-volatile (e.g. read-only memory (ROM)), flash memory, or any combination. System memory 1704 may include operating system 1705, one or more programming modules 1706, and may include a program data 1707. Operating system 1705, for example, may be suitable for controlling computing device 1700's operation. In one embodiment, programming modules 1706 may include image-processing module, machine learning module. Furthermore, embodiments of the disclosure may be practiced in conjunction with a graphics library, other operating systems, or any other application program and is not limited to any particular application or system. This basic configuration is illustrated in FIG. 17 by those components within a dashed line 1708.

Computing device 1700 may have additional features or functionality. For example, computing device 1700 may also include additional data storage devices (removable and/or non-removable) such as, for example, magnetic disks, optical disks, or tape. Such additional storage is illustrated in FIG. 17 by a removable storage 1709 and a non-removable storage 1710. Computer storage media may include volatile and non-volatile, removable and non-removable media implemented in any method or technology for storage of information, such as computer-readable instructions, data structures, program modules, or other data. System memory 1704, removable storage 1709, and non-removable storage 1710 are all computer storage media examples (i.e., memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 1700. Any such computer storage media may be part of device 1700. Computing device 1700 may also have input device(s) 1712 such as a keyboard, a mouse, a pen, a sound input device, a touch input device, a location sensor, a camera, a biometric sensor, etc. Output device(s) 1714 such as a display, speakers, a printer, etc. may also be included. The aforementioned devices are examples and others may be used.

Computing device 1700 may also contain a communication connection 1716 that may allow device 1700 to communicate with other computing devices 1718, such as over a network in a distributed computing environment, for example, an intranet or the Internet. Communication connection 1716 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both storage media and communication media.

As stated above, a number of program modules and data files may be stored in system memory 1704, including operating system 1705. While executing on processing unit 1702, programming modules 1706 (e.g., application 1720 such as a media player) may perform processes including, for example, one or more stages of methods, algorithms, systems, applications, servers, databases as described above. The aforementioned process is an example, and processing unit 1702 may perform other processes. Other programming modules that may be used in accordance with embodiments of the present disclosure may include machine learning applications.

Generally, consistent with embodiments of the disclosure, program modules may include routines, programs, components, data structures, and other types of structures that may perform particular tasks or that may implement particular abstract data types. Moreover, embodiments of the disclosure may be practiced with other computer system configurations, including hand-held devices, general purpose graphics processor-based systems, multiprocessor systems, microprocessor-based or programmable consumer electronics, application specific integrated circuit-based electronics, minicomputers, mainframe computers, and the like. Embodiments of the disclosure may also be practiced in distributed computing environments where tasks are performed by remote processing devices that are linked through a communications network. In a distributed computing environment, program modules may be located in both local and remote memory storage devices.

Furthermore, embodiments of the disclosure may be practiced in an electrical circuit comprising discrete electronic elements, packaged or integrated electronic chips containing logic gates, a circuit utilizing a microprocessor, or on a single chip containing electronic elements or microprocessors. Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

Embodiments of the disclosure, for example, may be implemented as a computer process (method), a computing system, or as an article of manufacture, such as a computer program product or computer readable media. The computer program product may be a computer storage media readable by a computer system and encoding a computer program of instructions for executing a computer process. The computer program product may also be a propagated signal on a carrier readable by a computing system and encoding a computer program of instructions for executing a computer process. Accordingly, the present disclosure may be embodied in hardware and/or in software (including firmware, resident software, micro-code, etc.). In other words, embodiments of the present disclosure may take the form of a computer program product on a computer-usable or computer-readable storage medium having computer-usable or computer-readable program code embodied in the medium for use by or in connection with an instruction execution system. A computer-usable or computer-readable medium may be any medium that can contain, store, communicate, propagate, or transport the program for use by or in connection with the instruction execution system, apparatus, or device.

The computer-usable or computer-readable medium may be, for example but not limited to, an electronic, magnetic, optical, electromagnetic, infrared, or semiconductor system, apparatus, device, or propagation medium. More specific computer-readable medium examples (a non-exhaustive list), the computer-readable medium may include the following: an electrical connection having one or more wires, a portable computer diskette, a random-access memory (RAM), a read-only memory (ROM), an erasable programmable read-only memory (EPROM or Flash memory), an optical fiber, and a portable compact disc read-only memory (CD-ROM). Note that the computer-usable or computer-readable medium could even be paper or another suitable medium upon which the program is printed, as the program can be electronically captured, via, for instance, optical scanning of the paper or other medium, then compiled, interpreted, or otherwise processed in a suitable manner, if necessary, and then stored in a computer memory.

Embodiments of the present disclosure, for example, are described above with reference to block diagrams and/or operational illustrations of methods, systems, and computer program products according to embodiments of the disclosure. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments of the disclosure have been described, other embodiments may exist. Furthermore, although embodiments of the present disclosure have been described as being associated with data stored in memory and other storage mediums, data can also be stored on or read from other types of computer-readable media, such as secondary storage devices, like hard disks, solid state storage (e.g., USB drive), or a CD-ROM, a carrier wave from the Internet, or other forms of RAM or ROM. Further, the disclosed methods' stages may be modified in any manner, including by reordering stages and/or inserting or deleting stages, without departing from the disclosure.

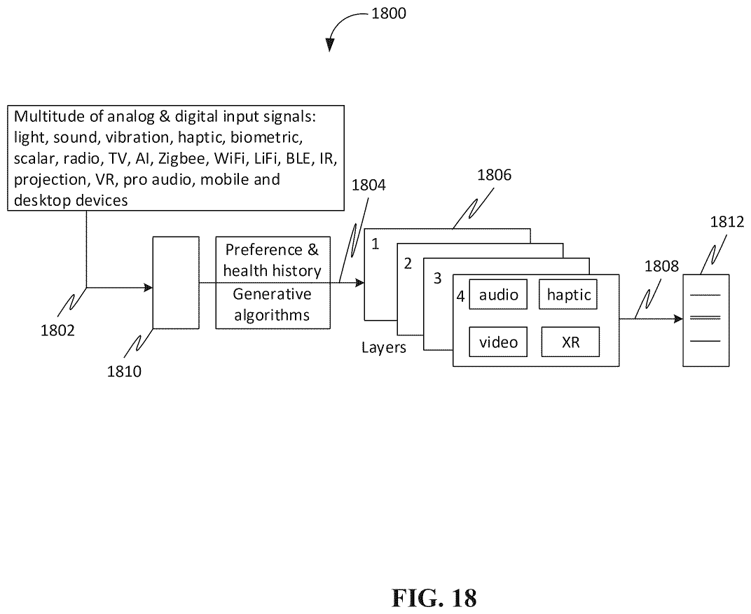

FIG. 18 is a flow diagram of a method 1800 for algorithmically generating personalized content by preset relationships for providing content to viewers for enhancing wellbeing of the viewers, in accordance with some embodiments. Further, the personalized content may be algorithmically generated by preset relationships or variables determined individually or in combination by AI, server, or other user generated broadcaster. Accordingly, at 1802 of the method 1800 multitude of analog & digital input signals may be received from light, sound, vibration, haptic, biometric, scalar, radio, TV, AI, Zigbee, WiFi, LiFi, BLE, IR, projection, VR, pro audio, mobile and desktop devices. Further, at 1804 of the method 1800, the multitude of analog & digital input signals may be processed based on preference and health history and generative algorithms using a local, remote, or web processing device 1810. Further, at 1806 of the method 1800, layers of Audio, Visual, Haptic, XR, or other Consumable Content are generated and Individually or in Combination is Uniquely placed or Augmented onto a base layer of which is determined size, layout, format, color, frequency, vibration, music key, tone, beat per minute, displacement, orientation, phase, amplitude, pulse width, modulation, prilling, sequence, geometry, network connection, and any relevant output. Further, at 1808 of the method 1800, the generated content may be transmitted to a Multitude of analog & digital output devices 1812 such as light, sound, vibration, haptic, biofeedback, scalar, radio, tv, binaural, multi-phase, Zigbee, WiFi, LiFi, BLE, IR, projection, IoT, Virtual Assistant, AI/VR, mobile and desktop devices in-home or in air/road travel environments. Further, user responses in real time, before, during & after experience are logged, categorized, and distributed to a database or ledger for instant use in the content generation loop. This data can be transferred or accessed by a wearable or otherwise connected device for secure access and programming.

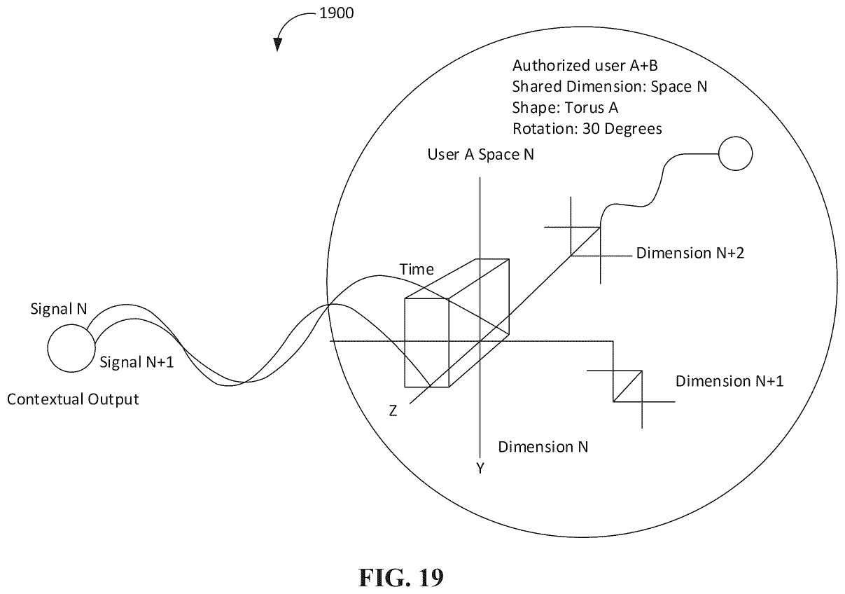

FIG. 19 is a visual representation 1900 of traveling of signals into a dimensional space for facilitating generating of the content for viewers for enhancing wellbeing of the viewers, in accordance with some embodiments. Further, real time signals associated with a broadcaster are filtered based on available outputs, preferences, and data preferences. Further, digital representations of the real time signals are generated and the output is based on, but not limited to, broadcaster and receiver's hardware and program functions comprising time, coordinate, rotation, prilling, torsion, cymatics, phase, amplitude, faces, vertices, and polygons. Further, evolving waveforms are placed in vectored dimensional space, extruding, deforming, and animating over time to represent a relationship context and an ongoing awareness of users' goals. Further, access and user relationships may be based on authorized identification and/or tokens which allows access to new layers, dimensions, times, and filters.

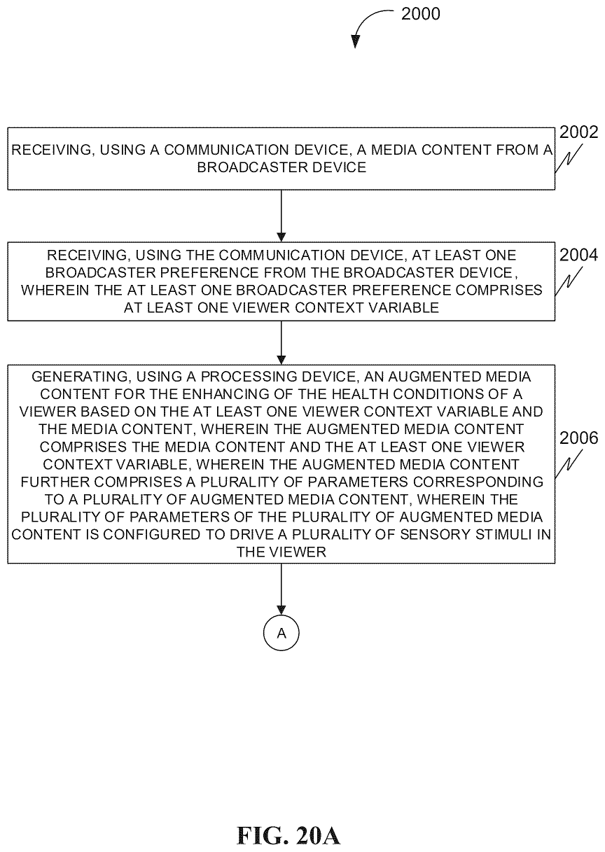

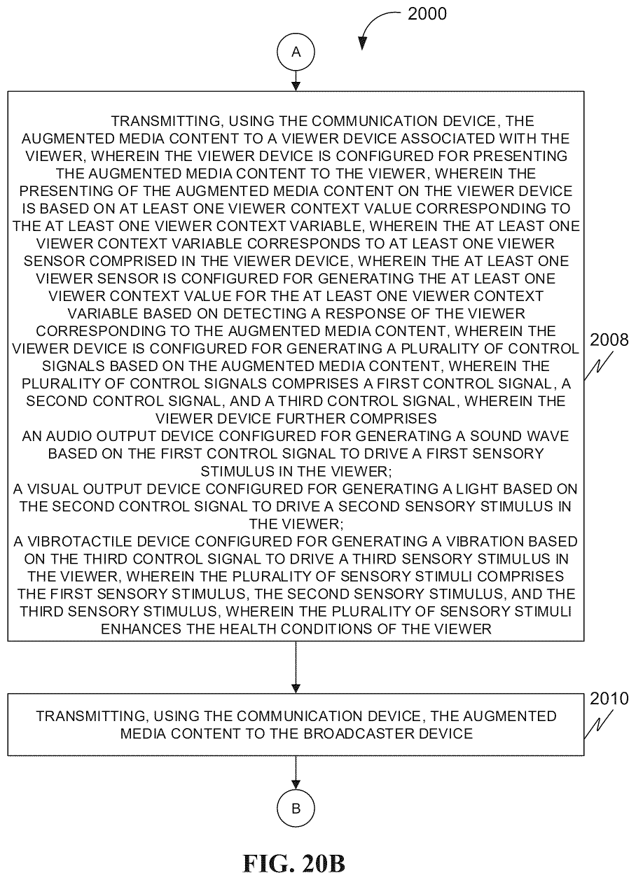

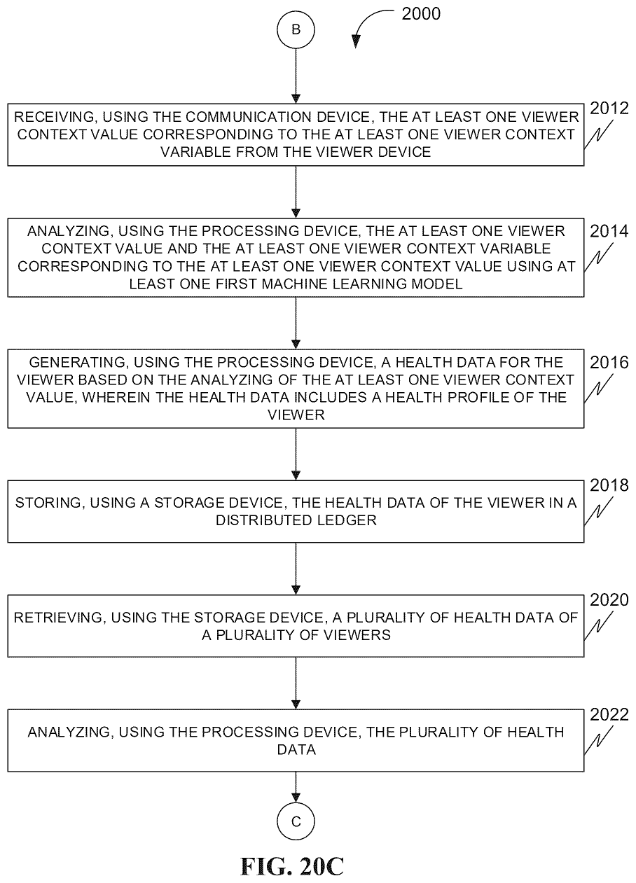

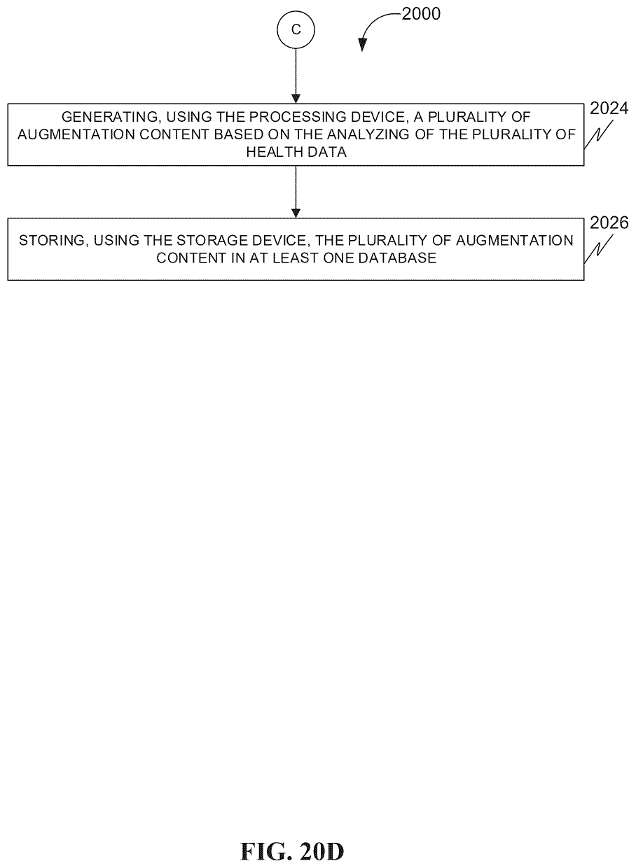

FIG. 20A, FIG. 20B, FIG. 20C, and FIG. 20D illustrates a flowchart of a method 2000 for providing content to viewers for enhancing health conditions of the viewers, in accordance with some embodiments. Further, the method 2000 may include a step 2002 of receiving, using a communication device 2402, a media content from a broadcaster device 2408. Further, the method 2000 may include a step 2004 of receiving, using the communication device 2402, one or more broadcaster preferences from the broadcaster device 2408. Further, the one or more broadcaster preferences include one or more viewer context variables. Further, the method 2000 may include a step 2006 of generating, using a processing device 2404, an augmented media content for the enhancing of the health conditions of a viewer based on the one or more viewer context variables and the media content. Further, the augmented media content includes the media content and the one or more viewer context variables. Further, the augmented media content further includes two or more parameters corresponding to two or more augmented media contents. Further, the two or more parameters of the two or more augmented media contents may be configured to drive two or more sensory stimuli in the viewer. Further, the method 2000 may include transmitting, using the communication device 2402, the augmented media content to a viewer device 2410 associated with the viewer. Further, the viewer device 2410 may be configured for presenting the augmented media content to the viewer. Further, the presenting of the augmented media content on the viewer device 2410 may be based on one or more viewer context values corresponding to the one or more viewer context variables. Further, the one or more viewer context variables correspond to one or more viewer sensors 2412 comprised in the viewer device 2410. Further, the one or more viewer sensors 2412 may be configured for generating the one or more viewer context values for the one or more viewer context variables based on detecting a response of the viewer corresponding to the augmented media content. Further, the viewer device 2410 may be configured for generating two or more control signals based on the augmented media content. Further, the two or more control signals may include a first control signal, a second control signal, and a third control signal. Further, the viewer device 2410 further may include an audio output device 2416 which may be configured for generating a sound wave based on the first control signal to drive a first sensory stimulus in the viewer. Further, the viewer device 2410 further may include a visual output device 2418 which may be configured for generating a light based on the second control signal to drive a second sensory stimulus in the viewer. Further, the viewer device 2410 further may include a vibrotactile device 2420 which may be configured for generating a vibration based on the third control signal to drive a third sensory stimulus in the viewer. Further, the two or more sensory stimuli include the first sensory stimulus, the second sensory stimulus, and the third sensory stimulus. Further, the two or more sensory stimuli enhance the health conditions of the viewer. Further, the method 2000 may include a step 2010 of transmitting, using the communication device 2402, the augmented media content to the broadcaster device 2408. Further, the method 2000 may include a step 2012 of receiving, using the communication device 2402, the one or more viewer context values corresponding to the one or more viewer context variables from the viewer device 2410. Further, the method 2000 may include a step 2014 of analyzing, using the processing device 2404, the one or more viewer context values and the one or more viewer context variables corresponding to the one or more viewer context values using one or more first machine learning models. Further, the method 2000 may include a step 2016 of generating, using the processing device 2404, a health data for the viewer based on the analyzing of the one or more viewer context values. Further, the health data may include a health profile of the viewer. Further, the method 2000 may include a step 2018 of storing, using a storage device 2406, the health data of the viewer in a distributed ledger. Further, the method 2000 may include a step 2020 of retrieving, using the storage device 2406, two or more health data of two or more viewers. Further, the method 2000 may include a step 2022 of analyzing, using the processing device 2404, the two or more health data. Further, the method 2000 may include a step 2024 of generating, using the processing device 2404, two or more augmentation contents based on the analyzing of the two or more health data. Further, the method 2000 may include a step 2026 of storing, using the storage device 2406, the two or more augmentation contents in one or more databases.

Further, in some embodiments, the generating of the augmented media content may include generating two or more programmable parameters corresponding to the two or more augmented media contents based on the one or more viewer context variables and the media content. Further, the two or more parameters include the two or more programmable parameters. Further, the generating of the augmented media content may include generating one or more instructions based on the two or more programmable parameters. Further, the one or more instructions may be configured to synchronize the vibration with the sound wave and the light. Further, the augmented media content further includes the one or more instructions.

Further, in some embodiments, the two or more parameters may include two or more frequencies of the sound wave. Further, the generating of the sound wave may include generating a first sound wave based on the first control signal. Further, the first sound wave may be characterized by a first frequency. Further, the generating of the sound wave may include generating a second sound wave based on the first control signal. Further, the second sound wave may be characterized by a second frequency. Further, the first frequency may be different from the second frequency. Further, the viewer receives the first sound wave through a first ear and the second sound wave through a second ear. Further, the enhancing of the health conditions of the viewer may be based on the receiving of the first sound wave through the first ear and the second sound wave through the second ear.

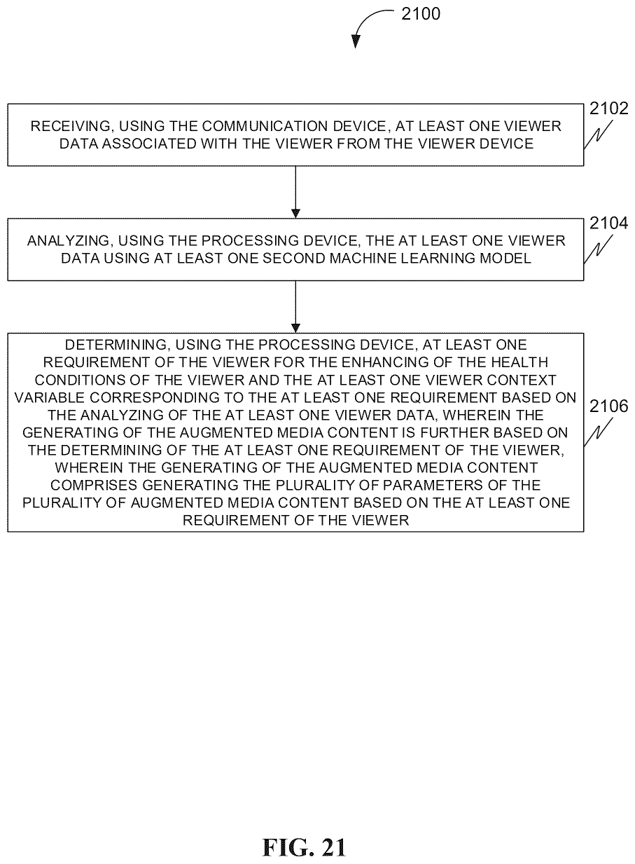

FIG. 21 illustrates a flowchart of a method 2100 for providing content to viewers for enhancing health conditions of the viewers including determining, using the processing device 2404, at least one requirement of the viewer for the enhancing of the health conditions of the viewer and the at least one viewer context variable corresponding to the at least one requirement, in accordance with some embodiments. Further, in some embodiments, the method 2100 further may include a step 2102 of receiving, using the communication device 2402, one or more viewer data associated with the viewer from the viewer device 2410. Further, in some embodiments, the method 2100 further may include a step 2104 of analyzing, using the processing device 2404, the one or more viewer data using one or more second machine learning models. Further, in some embodiments, the method 600 further may include a step 2106 of determining, using the processing device 2404, one or more requirements of the viewer for the enhancing of the health conditions of the viewer and the one or more viewer context variables corresponding to the one or more requirements based on the analyzing of the one or more viewer data. Further, the generating of the augmented media content may be further based on the determining of the one or more requirements of the viewer. Further, the generating of the augmented media content includes generating the two or more parameters of the two or more augmented media contents based on the one or more requirements of the viewer.

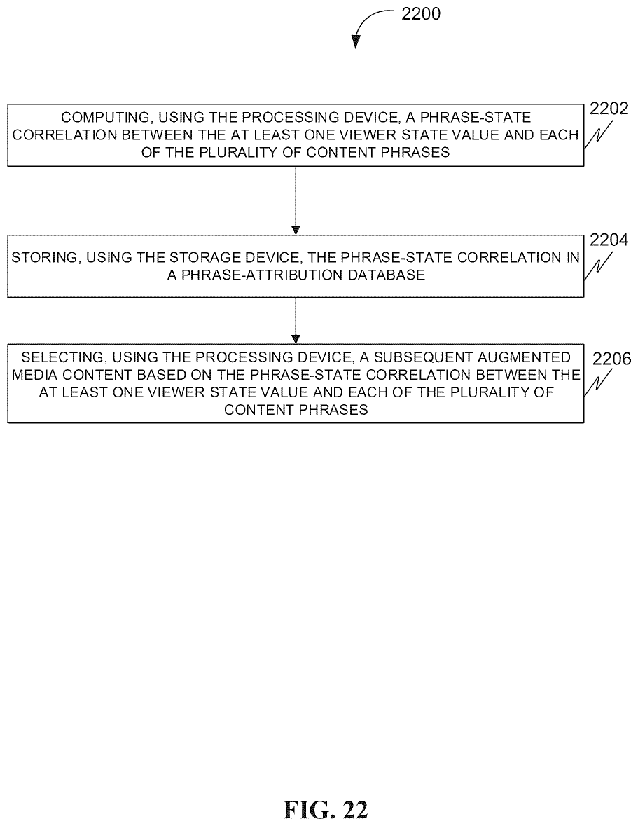

FIG. 22 illustrates a flowchart of a method 2200 for providing content to viewers for enhancing health conditions of the viewers including computing, using the processing device 2404, a phrase-state correlation between the at least one viewer state value and each of the plurality of content phrases, in accordance with some embodiments. Further, the generating of the augmented media content includes segmenting the media content into two or more content phrases. Further, the one or more viewer sensors 2412 may be further configured for measuring one or more viewer state values during the presenting of each of the two or more content phrases. Further, the one or more viewer context variables include one or more viewer state values. Further, in some embodiments, the method 2200 further may include a step 2202 of computing, using the processing device 2404, phrase-state correlation between the one or more viewer state values and each of the two or more content phrases. Further, in some embodiments, the method 2200 further may include a step 2204 of storing, using the storage device 2406, the phrase-state correlation in a phrase-attribution database. Further, in some embodiments, the method 2200 further may include a step 2206 of selecting, using the processing device 2404, a subsequent augmented media content based on the phrase-state correlation between the one or more viewer state values and each of the two or more content phrases.

In some embodiments, the method 2000 further may include generating, using the processing device 2404, two or more predefined parameters corresponding to two or more predetermined media contents based on the analyzing of the two or more health data. Further, the two or more predefined parameters may be configured to enhance two or more health conditions. Further, in some embodiments, the method further may include storing, using the storage device 2406, the two or more predefined parameters in the one or more databases.

In some embodiments, the method 2000 may include receiving, using the communication device 2402, a selection of at least one of the two or more of predefined parameters from the broadcaster device 2408, in accordance with some embodiments. Further, in some embodiments, the method 2000 further may include retrieving, using the storage device 2406, the two or more predefined parameters from the one or more databases based on the one or more broadcaster preferences. Further, in some embodiments, the method 2000 further may include transmitting, using the communication device 2402, the two or more predefined parameters to the broadcaster device 2408. Further, the broadcaster device 2408 may be configured to present the two or more predefined parameters. Further, in some embodiments, the method 2000 further may include receiving, using the communication device 2402, a selection of one or more of the two or more predefined parameters from the broadcaster device 2408. Further, the generating of the augmented media content may be based on the selection of one or more of the two or more predefined parameters. Further, the two or more parameters include one or more of the two or more predefined parameters. Further, the two or more augmented media contents include one or more of the two or more predetermined media contents.

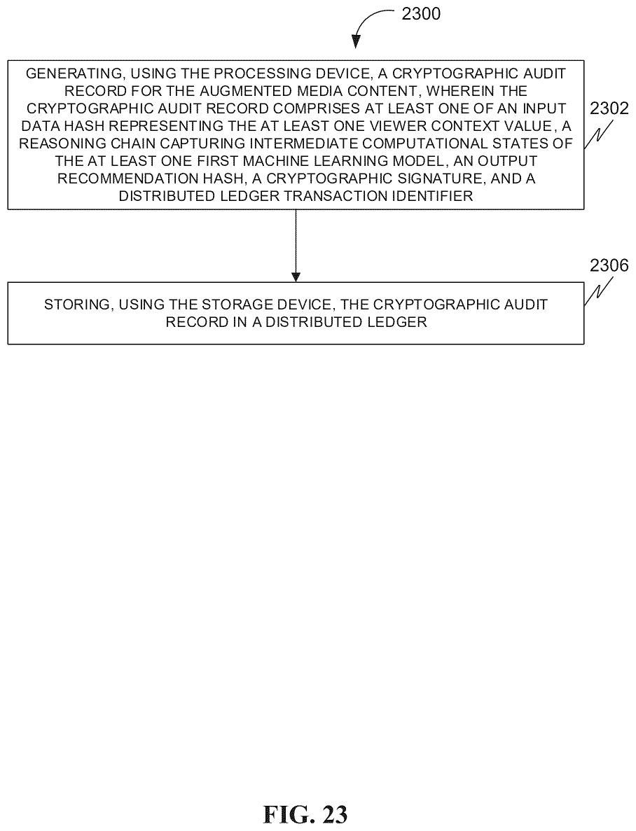

FIG. 23 illustrates a flowchart of a method 2300 for providing content to viewers for enhancing health conditions of the viewers including generating, using the processing device 2404, a cryptographic audit record for the augmented media content, in accordance with some embodiments. Further, in some embodiments, the method 2300 further may include a step 2302 of generating, using the processing device 2404, a cryptographic audit record for the augmented media content. Further, the cryptographic audit record comprises one or more of an input data hash representing the one or more viewer context value, a reasoning chain capturing intermediate computational states of the one or more first machine learning model, an output recommendation hash, a cryptographic signature, and a distributed ledger transaction identifier. Further, in some embodiments, the method 2300 further may include a step 2304 of storing, using the storage device 2406, the cryptographic audit record in a distributed ledger.

In some embodiments, the response of the viewer corresponding to the augmented media content includes a response of the viewer to one or more of the sound wave, the light, and the vibration. Further, the detecting of the response includes detecting a change in one or more of a physical state, a psychological state, and a biological state of the viewer in response to one or more of the sound wave, the light, and the vibration. Further, the generating 1002 of the one or more viewer context values for the one or more viewer context variables may be further based on the detecting of the change in one or more of the physical state, the psychological state, and the biological state of the viewer in response to one or more of the sound wave, the light, and the vibration.

In some embodiments, the phrase-state correlation comprises one or more of a content phrase identifier, a timestamp range corresponding to phrase presentation, a plurality of viewer state measurements captured during the timestamp range, a computed efficacy score indicating correlation strength between the phrase and a target viewer state, and a confidence interval for the efficacy score.

In some embodiments, the reasoning chain comprises one or more of a sequence of intermediate inference states, feature importance weights for each viewer context variable, attention scores indicating which input features influenced the recommendation, confidence scores for each intermediate decision, and a natural language explanatory text generated by the machine learning model.

Further, the one or more viewer sensors 2412 comprises a camera configured for detecting an eye state of the viewer comprising one or more of eyes-open and eyes-closed. Further, the camera is configured for selecting a sensing modality based on the detected eye state. Further, the sensing modality based on the eyes-opened comprises gaze tracking, pupillometry, and facial expression analysis. Further, the sensing modality based on the eyes-closed comprises facial micro-movement analysis, periorbital blood flow detection, and eyelid flutter analysis.

In some embodiments, the camera further comprises a near-infrared (NIR) illuminator. Further, the sensing modality based on the eyes-closed further comprises a functional near-infrared spectroscopy (fNIRS) for detecting prefrontal cortex activity patterns.

In some embodiments, the camera further comprises a video-based vital sign extraction configured for detecting subtle skin color variations to estimate heart rate. Further, the camera further comprises the video-based vital sign extraction configured for analyzing chest or shoulder movement to estimate respiratory rate. Further, the camera further comprises the video-based vital sign extraction configured for computing heart rate variability from inter-beat intervals. Further, the video-based vital sign extraction operates in both eyes-open and eyes-closed modes.

In some embodiments, the viewer device comprises a Tier 1 universal platform device. Further, the Tier 1 universal platform device comprises one or more of a smartphone, a tablet, and a laptop, audio output via built-in speakers or headphones, visual output via built-in display, and accelerometer-based motion detection. Further, the augmented media content may be optimized for the Tier 1 universal platform device capabilities.

In some embodiments, the viewer device comprises a Tier 2 screen-based platform device. Further, the Tier 2 screen-based platform device comprises all Tier 1 capabilities plus one or more of a dedicated visual display panel with controlled ambient lighting, camera-based viewer sensing, and environmental sensors. Further, the augmented media content optimized for the Tier 2 screen-based platform device further comprises coordinated ambient lighting patterns synchronized with the visual output.

In some embodiments, the viewer device comprises a Tier 3 hardware-integrated platform device. Further, the Tier 3 hardware-integrated platform device comprises all Tier 2 capabilities plus one or more of vibrotactile transducers embedded in seating or bed surface, dedicated biometric sensors including heart rate, respiration, and galvanic skin response, and haptic feedback devices. Further, the augmented media content optimized for the Tier 2 hardware-integrated platform device further comprises synchronized vibrotactile patterns, biometric-responsive adaptation, and multi-sensory immersive sequences.

In some embodiments, the method 2000 may further include establishing, using the processing device 2404, a viewer-specific safety profile comprising baseline physiological parameters and personal safety thresholds. Further, the method 2000 may further include comparing, using the processing device 2404, real-time viewer context values against the viewer-specific safety profile. Further, the method 2000 may further include generating, using the processing device 2404, graduated safety responses. Further, the graduated safety responses comprise a first level response reducing stimulus intensity, a second level response pausing content delivery, and a third level response terminating the session and alerting emergency contacts. Further, the safety monitoring operates as a parallel process independent of content generation.

In some embodiments, the analyzing using the one or more first machine learning models comprises federated learning. Further, model parameters may be transmitted to a plurality of viewer devices. Further, local training may occur on each viewer device using a local health data stored on a respective viewer device. Further, gradient updates may be transmitted from each of the plurality of viewer device to a central server. Further, a raw biometric data remains exclusively on the respective viewer device and may be never transmitted to the central server. Further, a global model may be updated based on aggregated gradients from the plurality of viewer devices, and updated global model parameters may be distributed to the plurality of viewer devices.

In some embodiments, the method 2000 further comprises applying, using the processing device 2404, a differential privacy to the gradient updates prior to transmission. Further, the differential privacy comprises adding calibrated noise to gradient values, verifying the gradient updates may not be reverse-engineered to reveal individual viewer data, and maintaining a privacy budget tracking cumulative privacy loss across training iterations.

In some embodiments, the method 2000 further comprises analyzing, using the processing device 2404, temporal patterns in historical viewer context values across a plurality of sessions. Further, the method 2000 further comprises generating, using the processing device 2404, a predictive health model for the viewer based on the temporal patterns. Further, the method 2000 further comprises predicting, using the processing device 2404, a future health state of the viewer based on current viewer context values and the predictive health model. Further, the method 2000 further comprises determining, using the processing device 2404, a predicted time horizon for the future health state. Further, the generating of the augmented media content may be further based on the predicting and the determining. Further, the augmented media content may be configured to prevent or mitigate the predicted future health state before the predicted future health state manifests. Further, the method 2000 further comprises updating, using the processing device 2404, the predictive health model based on a prediction accuracy.

In some embodiments, the predicting of the future health state comprises identifying precursor patterns in viewer context values. Further, the precursor patterns correspond to historically preceded adverse health states. Further, the predicting of the future health state comprises computing a probability of adverse state occurrence within the predicted time horizon. Further, the predicting of the future health state comprises determining intervention urgency based on the predicted severity and the predicted time horizon. Further, the predicting of the future health state comprises selecting proactive content intensity proportional to the intervention urgency.

In some embodiments, the generating of the at least one viewer context value comprises receiving a plurality of biometric signals from a plurality of sensor types. Further, the generating of the at least one viewer context value comprises applying sensor-specific confidence weights to each biometric signal based on signal quality metrics. Further, the generating of the at least one viewer context value comprises performing temporal alignment of asynchronous sensor streams to a common time reference. Further, the generating of the at least one viewer context value comprises computing a fused biometric state vector using weighted combination of aligned signals. Further, the generating of the at least one viewer context value comprises resolving conflicts between contradictory sensor readings using a hierarchical trust model wherein sensors with higher historical accuracy receive priority. Further, the generating of the at least one viewer context value comprises outputting the fused biometric state vector as the viewer context value.

In some embodiments, the hierarchical trust model comprises one or more of a sensor reliability score for each sensor type based on historical accuracy, a context-dependent weighting wherein certain sensors receive higher weight in specific context, a conflict resolution algorithm identifying contradictory readings and selects values based on reliability scores; and a dynamic recalibration mechanism updating reliability scores based on cross-validation between sensors.

In some embodiments, the enhancing of the health conditions comprises selecting from a plurality of therapeutic protocols based on the analyzed viewer context values and viewer-expressed wellness goals. Further, the plurality of therapeutic protocols comprises deficit-reduction protocols including one or more of an anxiety reduction protocol comprising synchronized low-frequency audio, warm-spectrum visual output, and rhythmic vibrotactile patterns at decreasing tempo. Further, the plurality of therapeutic protocols comprises a sleep induction protocol comprising binaural audio at delta frequencies (0.5-4 Hz), gradually dimming visual output following circadian patterns, and decelerating vibrotactile cadence synchronized with a target respiratory rate. Further, the plurality of therapeutic protocols comprises a pain management protocol comprising distraction-optimized visual content, gate-control vibrotactile stimulation at affected body regions, and entrainment audio at alpha frequencies. Further, plurality of therapeutic protocols comprises a trauma processing protocol comprising grounding sensory sequences, bilateral stimulation patterns alternating between left and right channels, and safe-state visual anchors.

In some embodiments, the plurality of therapeutic protocols comprises a stress reduction protocol comprising coherent breathing guidance synchronized across modalities, nature-based visual content, and parasympathetic-activating frequencies. Further, the plurality of therapeutic protocols comprises a positive-state enhancement protocol including a happiness elevation protocol comprising reward-associated visual content, major-key audio at elevated tempo (100-130 BPM), and pleasure-linked vibrotactile patterns calibrated to viewer preference history.

In some embodiments, the plurality of therapeutic protocols comprises a joy amplification protocol comprising peak-experience visual sequences, crescendo audio patterns building to euphoric release, and synchronized celebratory haptic feedback. Further, the plurality of therapeutic protocols comprises a gratitude cultivation protocol comprising memory-associated visual cues personalized to viewer history, heart-coherence audio entrainment, and warmth-simulating vibrotactile output.

In some embodiments, the plurality of therapeutic protocols comprises a motivation enhancement protocol comprising goal-visualization content, energizing audio frequencies in the beta range, and activating vibrotactile sequences with increasing intensity. Further, the plurality of therapeutic protocols comprises a creativity stimulation protocol comprising novel-association visual patterns, alpha-wave entrainment audio (8-12 Hz), and exploratory haptic sequences with varied textures.

In some embodiments, the plurality of therapeutic protocols comprises a social connection protocol comprising oxytocin-associated content patterns, prosocial audio cues, and bonding-simulating vibrotactile warmth. Further, the plurality of therapeutic protocols comprises a flow state induction protocol comprising challenge-matched content progression, focus-enhancing audio with minimal distraction, and distraction-blocking haptic anchors.

In some embodiments, the plurality of therapeutic protocols comprises a resilience-building protocol comprising mastery-reinforcing visual sequences showing progressive achievement, confidence-associated audio patterns, and grounding vibrotactile feedback.

In some embodiments, the happiness elevation protocol further comprises detecting baseline happiness indicators from viewer context values including facial micro-expression analysis detecting genuine smile markers, voice prosody patterns indicating positive affect, and physiological arousal markers consistent with positive emotional states. Further, the happiness elevation protocol further comprises selecting content elements correlated with elevated happiness markers in the viewer's historical session data. Further, the happiness elevation protocol further comprises real-time adjustment of protocol parameters based on detected happiness response trajectory. Further, the happiness elevation protocol further comprises storing happiness-correlated content preferences for future session optimization. Further, the happiness elevation protocol further targets activation of neural reward pathways through coordinated multi-sensory stimulation.

In some embodiments, the positive-state enhancement protocols further comprise a sustainability phase following peak positive state achievement configured to maintain elevated state. Further, the positive-state enhancement protocols further comprise gradual parameter modulation during the sustainability phase to maintain elevated state without habituation or hedonic adaptation. Further, the positive-state enhancement protocols further comprise anchor establishment linking the positive state to user-controllable cues reproducible outside the session. Further, the positive-state enhancement protocols further comprise transition sequence preparing viewer for session conclusion while preserving positive affect. Further, the positive-state enhancement protocols further comprise post-session micro-interventions delivered via the viewer device to extend positive state duration.

In some embodiments, the method 2000 may further include generating, using the processing device 2404, a personalized happiness profile based on accumulated viewer response data across multiple sessions. Further, the method 2000 may further include identifying, using the processing device 2404, viewer-specific happiness triggers across sensory modalities including visual preferences, audio preferences, and vibrotactile preferences. Further, the method 2000 may further include weighting, using the processing device 2404, therapeutic protocol parameters according to the personalized happiness profile. Further, the method 2000 may further include updating, using the processing device 2404, the personalized happiness profile based on session outcomes and viewer feedback. Further, the method 2000 may further include cross-referencing, using the processing device 2404, the personalized happiness profile with population-level happiness correlates to identify potentially effective untested interventions.

In some embodiments, the method 2000 may further include detecting, using the processing device 2404, unavailability or malfunction of at least one viewer sensor or output device. Further, the method 2000 may further include determining, using the processing device 2404, a degraded sensing or output configuration based on remaining available components. Further, the method 2000 may further include selecting, using the processing device 2404, an alternative augmented media content optimized for the degraded configuration. Further, the method 2000 may further include recalculating, using the processing device 2404, expected therapeutic efficacy for the degraded configuration. Further, the method 2000 may further include notifying, using the processing device 2404, the viewer of degraded operation mode and adjusted expectations. Further, the method 2000 may further include maintaining, using the processing device 2404, a therapeutic benefit within a predetermined minimum threshold despite sensor or output unavailability.

In some embodiments, the alternative augmented media content comprises compensatory intensification of available output modalities to offset unavailable modalities. Further, the alternative augmented media content comprises modified sensing strategies using available sensors to estimate parameters normally captured by unavailable sensors. Further, the alternative augmented media content comprises reduced therapeutic scope focusing on objectives achievable with degraded configuration. Further, the alternative augmented media content comprises graceful capability restoration when previously unavailable components become available.

In some embodiments, the method 2000 may further include storing, using the storage device 2406, a session state data in a viewer-associated persistent storage upon session termination. Further, the method 2000 may further include retrieving, using the storage device 2406, the session state data upon subsequent session initiation. Further, the method 2000 may further include computing, using the processing device 2404, a longitudinal health trajectory across a plurality of sessions. Further, the method 2000 may further include computing, using the processing device 2404, identifying trends and patterns in the longitudinal health trajectory. Further, the method 2000 may further include adapting, using the processing device 2404, the augmented media content parameters based on the longitudinal trajectory to optimize long-term therapeutic outcomes. Further, the method 2000 may further include generating, using the processing device 2404, a therapeutic progress metric quantifying improvement over the plurality of sessions. Further, the method 2000 may further include presenting, using the processing device 2404, the therapeutic progress metric to the viewer.

In some embodiments, the longitudinal health trajectory comprises baseline measurements from initial sessions, periodic progress measurements at defined intervals, trend analysis indicating improvement, plateau, or regression, predicted future trajectory based on current trend, and recommended therapeutic adjustments to optimize the predicted future trajectory.

In some embodiments, the method 2000 may further include generating, using the processing device 2404, a digital twin model of the viewer based on accumulated health data, viewer context values, and historical response patterns; simulating viewer responses to a plurality of candidate augmented media contents using the digital twin model prior to actual delivery. Further, the method 2000 may further include ranking, using the processing device 2404, the plurality of candidate augmented media contents based on simulated therapeutic efficacy. Further, the method 2000 may further include selecting, using the processing device 2404, an optimal augmented media content based on simulation results. Further, the method 2000 may further include delivering, using the communication device 2402, the selected optimal augmented media content to the viewer device. Further, the method 2000 may further include measuring, using the processing device 2404, an actual viewer response to the delivered content. Further, the method 2000 may further include computing, using the processing device 2404, a simulation accuracy by comparing simulated response to actual response. Further, the method 2000 may further include updating, using the processing device, the digital twin model based on simulation accuracy to improve future predictions.

In some embodiments, the digital twin model comprises a physiological response model predicting biometric changes in response to stimuli, a psychological state model predicting emotional and cognitive responses, a preference model predicting content preferences and engagement a temporal dynamics model predicting response timing and duration, and an interaction model predicting cross-modal effects between simultaneous stimuli.

In some embodiments, the reasoning chain further comprises natural language explanations generated for each therapeutic recommendation in vocabulary accessible to non-technical viewers, counterfactual explanations indicating what changes in viewer context values would alter the recommendation, confidence intervals for each recommendation component, citations to supporting patterns in the viewer's historical health data, and comparison to population-level response patterns for similar viewer profiles.

In some embodiments, the method 2000 may further include generating, using the processing device 2404, a viewer-facing explanation summary suitable for display on the viewer device. Further, the method 2000 may further include providing, using the processing device 2404, explanation detail levels selectable by the viewer from summary to comprehensive. Further, the method 2000 may further include enabling, using the processing device 2404, viewer query of specific recommendation factors. and Further, the method 2000 may further include storing, using the storage device 2406, viewer explanation interactions to improve future explanation generation.

In some embodiments, the method 2000 may further include presenting, using the communication device 2402, a calibration sequence to the viewer at session initiation. Further, the calibration sequence comprises a series of standardized stimuli designed to establish baseline responses. Further, the method 2000 may further include measuring, using the processing device 2404, baseline viewer context values during the calibration sequence. Further, the method 2000 may further include computing, using the processing device 2404, viewer-specific normalization parameters based on the baseline measurements. Further, the method 2000 may further include applying, using the processing device 2404, normalization to subsequent viewer context values throughout the session to account for individual variation. Further, the method 2000 may further include detecting, using the processing device 2404, baseline drift during extended sessions; and periodically recalibrating based on detected baseline drift.

In some embodiments, the calibration sequence comprises a visual calibration component measuring pupil response to standardized light stimuli, an audio calibration component measuring physiological response to standardized sound stimuli, a vibrotactile calibration component measuring sensitivity to standardized vibration patterns, and a combined calibration component measuring cross-modal response characteristics.

In some embodiments, the viewer device is associated with a plurality of viewers simultaneously. Further, the method 2000 may further include receiving, using the communication device 2402, viewer context values from each of the plurality of viewers via respective viewer sensors. Further, the method 2000 may further include computing, using the processing device 2404, a group state vector representing collective state of the plurality of viewers. Further, the method 2000 may further include identifying, using the processing device 2404, outlier individual states deviating significantly from the group state. Further, the method 2000 may further include generating, using the processing device 2404, synchronized augmented media content optimized for collective therapeutic benefit of the plurality of viewers. Further, the method 2000 may further include balancing individual viewer needs with group coherence in content selection. Further, the method 2000 may further include providing, using the processing device 2404, individualized content modifications for outlier viewers while maintaining group synchronization. Further, the method 2000 may further include computing, using the processing device 2404, group therapy efficacy metrics.

In some embodiments, the method 2000 may further include detecting, using the processing device 2404, social interaction patterns between the plurality of viewers. Further, the method 2000 may further include modifying, using the processing device 2404, content to enhance positive social interactions. Further, the method 2000 may further include identifying, using the processing device 2404, negative social dynamics detected to mitigate the negative social dynamics through viewer context analysis. Further, the method 2000 may further include generating, using the processing device 2404, post-session reports for group therapy facilitators.

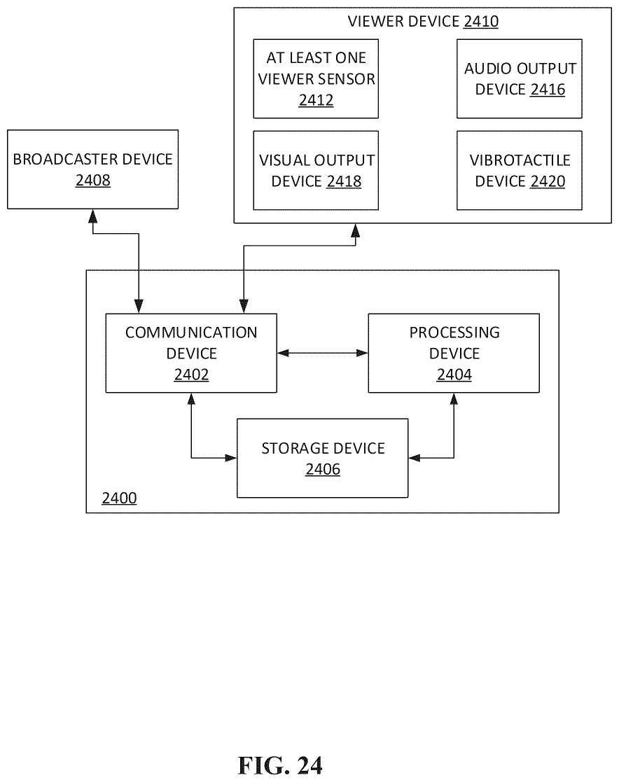

FIG. 24 illustrates a block diagram of a system 2400 for providing content to viewers for enhancing health conditions of the viewers, in accordance with some embodiments. Further, the system 2400 may include a communication device 2402. Further, the communication device 2402 may be configured for receiving a media content from a broadcaster device 2408. Further, the communication device 2402 may be configured for receiving one or more broadcaster preferences from the broadcaster device 2408. Further, the one or more broadcaster preferences include one or more viewer context variables. Further, the communication device 2402 may be configured for transmitting an augmented media content to a viewer device 2410 associated with a viewer. Further, the viewer device 2410 may be configured for presenting the augmented media content to the viewer. Further, the presenting of the augmented media content on the viewer device 2410 may be based on one or more viewer context values corresponding to the one or more viewer context variables. Further, the one or more viewer context variables correspond to one or more viewer sensors 2412 comprised in the viewer device 2410. Further, the one or more viewer sensors 2412 may be configured for generating the one or more viewer context values for the one or more viewer context variables based on detecting a response of the viewer corresponding to the augmented media content. Further, the viewer device 2410 may be configured for generating two or more control signals based on the augmented media content. Further, the two or more control signals may include a first control signal, a second control signal, and a third control signal. Further, the viewer device 2410 further may include an audio output device 2416 which may be configured for generating a sound wave based on the first control signal to drive a first sensory stimulus in the viewer. Further, the viewer device 2410 further may include a visual output device 2418 which may be configured for generating a light based on the second control signal to drive a second sensory stimulus in the viewer. Further, the viewer device 2410 further may include a vibrotactile device 2420 which may be configured for generating a vibration based on the third control signal to drive a third sensory stimulus in the viewer. Further, the two or more sensory stimuli include the first sensory stimulus, the second sensory stimulus, and the third sensory stimulus. Further, two or more sensory stimuli enhance the health conditions of the viewer. Further, the communication device 2402 may be configured for transmitting the augmented media content to the broadcaster device 2408. Further, the communication device 2402 may be configured for receiving the one or more viewer context values corresponding to the one or more viewer context variables from the viewer device 2410. Further, the system 2400 may include a processing device 2404 communicatively coupled with the communication device 2402. Further, the processing device 2404 may be configured for generating the augmented media content for the enhancing of the health conditions of a viewer based on the one or more viewer context variables and the media content. Further, the augmented media content includes the media content and the one or more viewer context variables. Further, the augmented media content further includes two or more parameters corresponding to two or more augmented media contents. Further, the two or more parameters of the two or more augmented media contents may be configured to drive the two or more sensory stimuli in the viewer. Further, the processing device 2404 may be configured for analyzing the one or more viewer context values and the one or more viewer context variables corresponding to the one or more viewer context values using one or more first machine learning models. Further, the processing device 2404 may be configured for generating a health data for the viewer based on the analyzing of the one or more viewer context values. Further, the health data may include a health profile of the viewer. Further, the processing device 2404 may be configured for analyzing two or more health data. Further, the processing device 2404 may be configured for generating two or more augmentation contents based on the analyzing of the two or more health data. Further, the system 2400 may include a storage device 2406 communicatively coupled with the processing device 2404. Further, the storage device 2406 may be configured for storing the health data of the viewer in a distributed ledger. Further, the storage device 2406 may be configured for retrieving the two or more health data of two or more viewers. Further, the storage device 2406 may be configured for storing the two or more augmentation contents in one or more databases.

Further, in some embodiments, the generating of the augmented media content may include generating two or more programmable parameters corresponding to the two or more augmented media based on the one or more viewer context variables and the media content. Further, the two or more parameters include the two or more programmable parameters. Further, the generating of the augmented media content may include generating one or more instructions based on the two or more programmable parameters. Further, the one or more instructions may be configured to synchronize the vibration with the sound wave and the light. Further, the augmented media content further includes the one or more instructions.

Further, in some embodiments, the two or more parameters may include two or more frequencies of the sound wave. Further, the generating of the sound wave may include generating a first sound wave based on the first control signal. Further, the first sound wave may be characterized by a first frequency. Further, the generating of the sound wave may include generating a second sound wave based on the first control signal. Further, the second sound wave may be characterized by a second frequency. Further, the first frequency may be different from the second frequency. Further, the viewer receives the first sound wave through a first ear and the second sound wave through a second ear. Further, the enhancing of the of the health conditions of the viewer may be based on the receiving of the first sound wave through the first ear and the second sound wave through the second ear.

Further, in some embodiments, the communication device 2402 may be configured for receiving one or more viewer data associated with the viewer from the viewer device. Further, the processing device 2404 may be further configured for analyzing the one or more viewer data using one or more second machine learning models. Further, the processing device 2404 may be further configured for determining one or more requirements of the viewer for the enhancing of the health conditions of the viewer and the one or more viewer context variables corresponding to the one or more requirements based on the analyzing of the one or more viewer data. Further, the generating of the augmented media content may be further based on the determining of the one or more requirements of the viewer. Further, the generating of the augmented media content includes generating the two or more parameters of the two or more augmented media contents based on the one or more requirements of the viewer.

In some embodiments, the processing device 2404 may be further configured for generating two or more predefined parameters corresponding to two or more predetermined media contents based on the analyzing of the two or more health data. Further, the two or more predefined parameters may be configured to enhance two or more health conditions. Further, the storage device 2406 may be further configured for storing the two or more predefined parameters in the one or more databases.

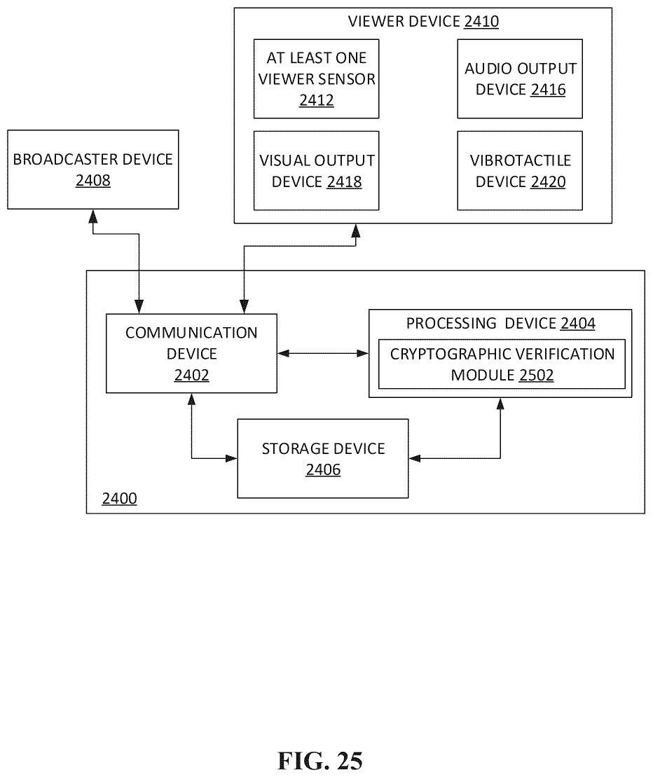

FIG. 25 illustrates a block diagram of the system 2400 for providing content to viewers for enhancing health conditions of the viewers, in accordance with some embodiments. Further, the processing device 2404 includes a cryptographic verification module 2502. Further, the cryptographic verification module 2502 may be configured for retrieving a cryptographic audit record from a distributed ledger. Further, the cryptographic verification module 2502 may be further configured for verifying a cryptographic signature based on the retrieving of the cryptographic audit record. Further, the cryptographic audit record comprises the cryptographic signature. Further, the cryptographic verification module 2502 may be further configured for reconstructing a reasoning chain based on the verifying. Further, the reasoning chain indicates intermediate computational states of the one or more first machine learning models. Further, the cryptographic verification module 2502 may be further configured for generating a verification report based on the reconstruction of the reasoning chain and the verifying of the cryptographic audit record. Further, the verification report may confirm an accordance of the augmented media content with the reasoning chain.

Further, in some embodiments, the storage device 2406 may be further configured for retrieving the two or more predefined parameters from the one or more databases based on the one or more broadcaster preferences. Further, the communication device 2402 may be further configured for transmitting the two or more predefined parameters to the broadcaster device 2408. Further, the broadcaster device 2408 may be configured to present the two or more predefined parameters. Further, the communication device 2402 may be further configured for receiving a selection of one or more of the two or more predefined parameters from the broadcaster device 2408. Further, the generating of the augmented media content may be based on the selection of one or more of the two or more predefined parameters. Further, the two or more parameters include one or more of the two or more predefined parameters. Further, the two or more augmented media contents include one or more of the two or more predetermined media contents.

In some embodiments, the response of the viewer corresponding to the augmented media content includes a response of the viewer to one or more of the sound wave, the light, and the vibration. Further, the detecting of the response includes detecting a change in one or more of a physical state, a psychological state, and a biological state of the viewer in response to one or more of the sound wave, the light, and the vibration. Further, the generating of the one or more viewer context values for the one or more viewer context variables may be further based on the detecting of the change in one or more of the physical state, the psychological state, and the biological state of the viewer in response to one or more of the sound wave, the light, and the vibration.

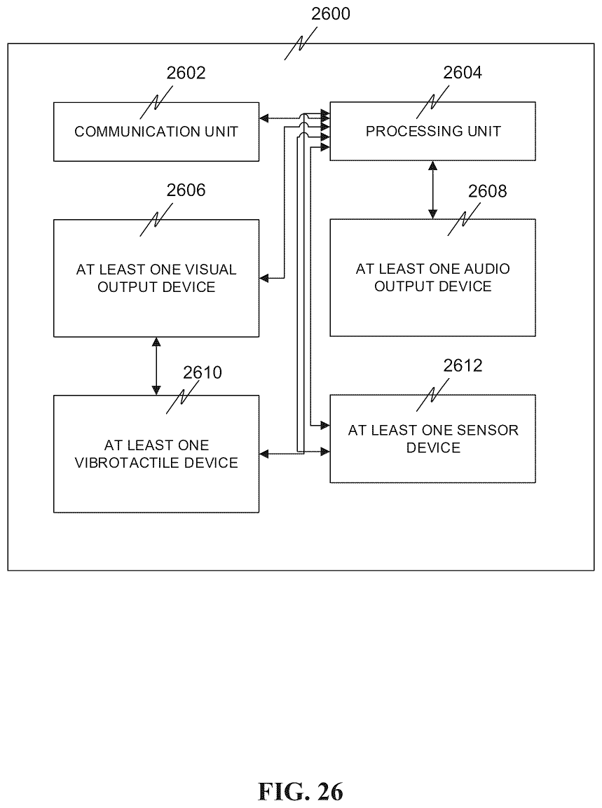

FIG. 26 illustrates a block diagram of the device 2600 for providing content to viewers for enhancing health conditions of the viewers, in accordance with some embodiments. Further, the device 2600 may include a communication unit 2602. Further, the communication unit 2602 may be configured for receiving one or more media signals from a broadcaster device. Further, the one or more media signal corresponds to a media content. Further, the communication unit 2602 may be configured for receiving one or more broadcaster preferences from the broadcaster device. Further, the one or more broadcaster preferences include one or more viewer context variables. Further, the device 2600 may include a processing unit 2604 communicatively coupled with the communication unit 2602. Further, the processing unit 2604 may be configured for generating an augmented media signal for the enhancing of the health conditions of a viewer based on the one or more media signals and the one or more broadcaster preferences. Further, the processing unit 2604 may be configured for generating two or more control signals based on the augmented media signal. Further, the device 2600 may include one or more visual output devices 2606 communicatively coupled with the processing unit 2604. Further, the visual output device 2606 may be configured for generating a light based on a first control signal. Further, the two or more control signals include the first control signal. Further, the viewer may be associated with the one or more visual output devices 2606. Further, the device 2600 may include one or more audio output devices 2608 communicatively coupled with the processing unit 2604. Further, the one or more audio output devices 2608 may be configured for generating one or more sound waves based on a second control signal. Further, the two or more control signals further include the second control signal. Further, the viewer may be further associated with the one or more audio output devices 2608. Further, the device 2600 may include one or more vibrotactile devices 2610 communicatively coupled with the processing unit 2604. Further, the one or more vibrotactile devices 2610 may be configured for generating one or more vibrations based on a third control signal. Further, the two or more control signals further include the third control signal. Further, the viewer may be further associated with the one or more vibrotactile devices 2610. Further, the light, the one or more sound waves, and the one or more vibrations enhance the health conditions of the viewer. Further, the device 2600 may include one or more sensor devices 2612 communicatively coupled with the processing unit 2604. Further, the one or more sensor devices 2612 may be configured for detecting one or more responses of the viewer to one or more of the light, the one or more sound waves, and the one or more vibrations. Further, the one or more sensor devices 2612 may be configured for generating one or more viewer context values for the one or more viewer context variables based on the detecting of the one or more responses. Further, the generating of the augmented media signal for the enhancing of the health conditions may be further based on the one or more viewer context values.

In some embodiments, the one or more vibrotactile devices 2610 include a bed which may be configured to vibrate based on the third control signal. Further, the bed includes one or more of water and a foam.

In some embodiments, the detecting of the response includes detecting a change in one or more of a physical state, a psychological state, and a biological state of the viewer in response to one or more of the sound wave, the light, and the vibration. Further, the. Further, the generating of the one or more viewer context values for the one or more viewer context variables may be further based on the detecting of the change in one or more of the physical state, the psychological state, and the biological state of the viewer in response to one or more of the sound wave, the light, and the vibration.

In some embodiments, the one or more sensor devices 2612 may be further configured for generating one or more images of the viewer experiencing one or more of the light, the one or more sound waves, and the one or more vibrations. Further, the detecting of the response of the viewer may be further based on the one or more images.

In some embodiments, the one or more sensor devices 2612 comprises a visible spectrum camera configured for performing facial expression analysis during an eyes-open state of the viewer. Further, the one or more sensor devices 2612 further comprises a visible spectrum camera configured for performing physiological monitoring during an eyes-closed state of the viewer. Further, the one or more sensor devices 2612 further comprises a mode controller configured for switching between the performing of the facial expression analysis and the performing of the physiological monitoring based on an eye state of the viewer. Further, the mode controller ensures continuous biometric monitoring regardless of the eye state of the viewer.

In some embodiments, the generating of the augmented media signal includes generating two or more parameters corresponding to the light, the one or more sound waves, and the one or more vibrations based on the one or more media signals and the one or more broadcaster preferences. Further, the generating of the two or more control signals may be based on the two or more parameters.

In some embodiments, the one or more sound waves may be configured to drive a first sensory stimulus in the viewer. Further, the light may be configured to drive a second sensory stimulus in the viewer. Further, the one or more vibrations may be configured to drive a third sensory stimulus in the viewer. Further, the first sensory stimulus, the second sensory stimulus, and the third sensory stimulus enhance the health conditions of the viewer.

In some embodiments, the device 2600 further comprises a safety monitoring module independent from the processing unit 2604. Further, the safety monitoring module may be configured for continuously monitoring viewer physiological parameters. Further, the safety monitoring module may be configured for comparing monitored parameters against predetermined safety thresholds. Further, the safety monitoring module may be configured for generating a safety intervention signal based on monitored parameter exceeding the predetermined safety threshold. Further, the safety intervention signal may cause immediate modification or cessation of sensory stimuli output independent of operation of the processing unit 2604.

In some embodiments, the safety monitoring module further comprises a dedicated NIR sensor for continuous pulse oximetry. Further, the safety monitoring module comprises a motion sensor for detecting viewer distress movements. Further, the safety monitoring module comprises an audio sensor for detecting viewer vocalizations indicating distress. Further, the safety monitoring module operates on dedicated hardware separate from a primary device processing.

In some embodiments, the one or more sounds wave corresponds to a binaural audio.

In some embodiments, the light includes a pulsating light.

In some embodiments, the one or more sensor devices 2612 may be implanted on the viewer. Further, the one or more viewer context values include one or more physiological values of the viewer.

In some embodiments, the enhancing of the health conditions of the viewer includes reducing one or more of a mental issue of the viewer, physical issues of the viewer, and an emotional issue of the viewer.

In some embodiments, the enhancing of the health conditions of the viewer includes stimulating a nervous system of the viewer.

In some embodiments, the enhancing of the health conditions of the viewer corresponds to one or more of relieving pain, releasing trauma, reducing stress, reducing anxiety, improving sleep, and improving focus.

In some embodiments, the enhancing of the health conditions of the viewer includes stimulating a harmonic state of the viewer.

In some embodiments, the enhancing of the health conditions of the viewer enhances a sensory integration and a motor skill of the viewer.

In some embodiments, the enhancing of the health conditions of the viewer corresponds to a psychedelic therapy.

In some embodiments, the enhancing of the health conditions of the viewer includes activating one or more regions of a brain of the viewer.

In some embodiments, a non-transitory computer-readable medium storing instructions that, when executed by a processor, cause the processor to perform operations comprising receiving a media content from a broadcaster device. Further, the operations comprise receiving at least one broadcaster preference comprising at least one viewer context variable. Further, the operations comprise generating an augmented media content for enhancing health conditions of a viewer based on the viewer context variable and the media content. Further, the operations comprise transmitting the augmented media content to a viewer device. Further, the operations comprise receiving viewer context values from the viewer device. Further, the operations comprise analyzing the viewer context values using at least one machine learning model. Further, the operations comprise generating health data based on the analyzing.

In some embodiments, the operations further comprise segmenting the media content into a plurality of content phrases. Further, the operations comprise computing phrase-state correlations between content phrases and viewer states. Further, the operations comprise selecting subsequent content based on stored phrase-state correlations.

In some embodiments, the operations further comprise generating a cryptographic audit record comprising a reasoning chain of the machine learning model. Further, the operations comprise storing the cryptographic audit record in a distributed ledger.

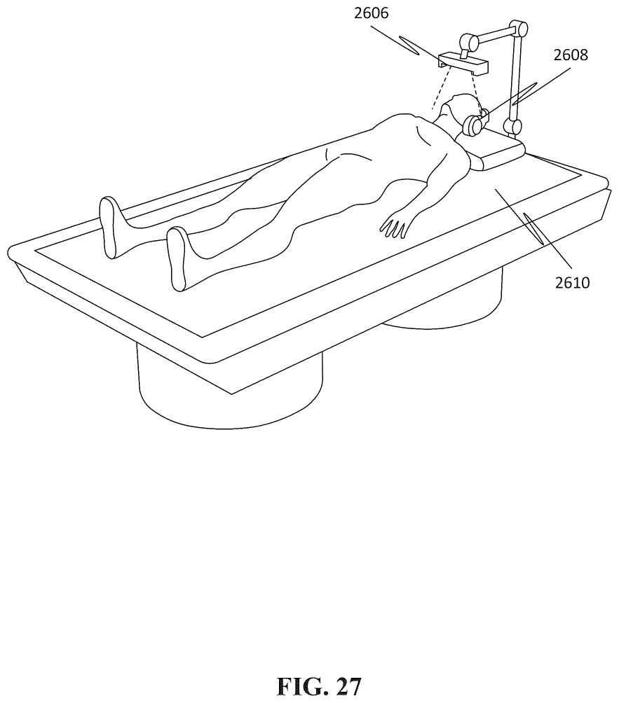

FIG. 27 illustrates a therapeutic application of the device 2600 for providing content to viewers for enhancing health conditions of the viewers, in accordance with some embodiments. Further, FIG. 27 shows the one or more visual output devices 2606, the one or more audio output devices 2608, and the one or more vibrotactile devices 2610.

Although the invention has been explained in relation to its preferred embodiment, it is to be understood that many other possible modifications and variations can be made without departing from the spirit and scope of the invention as hereinafter claimed.

What is claimed is:

1. A method for providing content to viewers for enhancing health conditions of the viewers, the method comprising:
receiving, using a communication device, a media content from a broadcaster device;
receiving, using the communication device, at least one broadcaster preference from the broadcaster device, wherein the at least one broadcaster preference comprises at least one viewer context variable;
generating, using a processing device, an augmented media content for the enhancing of the health conditions of a viewer based on the at least one viewer context variable and the media content, wherein the augmented media content comprises the media content and the at least one viewer context variable, wherein the augmented media content further comprises a plurality of parameters corresponding to a plurality of augmented media content, wherein the plurality of parameters of the plurality of augmented media content is configured to drive a plurality of sensory stimuli in the viewer;
transmitting, using the communication device, the augmented media content to a viewer device associated with the viewer, wherein the viewer device is configured for presenting the augmented media content to the viewer, wherein the presenting of the augmented media content on the viewer device is based on at least one viewer context value corresponding to the at least one viewer context variable, wherein the at least one viewer context variable corresponds to at least one viewer sensor comprised in the viewer device, wherein the at least one viewer sensor is configured for generating the at least one viewer context value for the at least one viewer context variable based on detecting a response of the viewer corresponding to the augmented media content, wherein the viewer device is configured for generating a plurality of control signals based on the augmented media content, wherein the plurality of control signals comprises a first control signal, a second control signal, and a third control signal, wherein the viewer device further comprises:
an audio output device configured for generating a sound wave based on the first control signal to drive a first sensory stimulus in the viewer;
a visual output device configured for generating a light based on the second control signal to drive a second sensory stimulus in the viewer;
and
a vibrotactile device configured for generating a vibration based on the third control signal to drive a third sensory stimulus in the viewer, wherein the plurality of sensory stimuli comprises the first sensory stimulus, the second sensory stimulus, and the third sensory stimulus, wherein the plurality of sensory stimuli enhances the health conditions of the viewer;
transmitting, using the communication device, the augmented media content to the broadcaster device;
receiving, using the communication device, the at least one viewer context value corresponding to the at least one viewer context variable from the viewer device;
analyzing, using the processing device, the at least one viewer context value and the at least one viewer context variable corresponding to the at least one viewer context value using at least one first machine learning model;
generating, using the processing device, a health data for the viewer based on the analyzing of the at least one viewer context value, wherein the health data includes a health profile of the viewer;
storing, using a storage device, the health data of the viewer in a distributed ledger;
retrieving, using the storage device, a plurality of health data of a plurality of viewers;
analyzing, using the processing device, the plurality of health data;
generating, using the processing device, a plurality of augmentation content based on the analyzing of the plurality of health data; and
storing, using the storage device, the plurality of augmentation content in at least one database.

2. The method of claim 1, wherein the generating of the augmented media content comprises:
generating a plurality of programmable parameters corresponding to the plurality of augmented media content based on the at least one viewer context variable and the media content, wherein the plurality of parameters comprises the plurality of programmable parameters; and
generating at least one instruction based on the plurality of programmable parameters, wherein the at least one instruction is configured to synchronize the vibration with the sound wave and the light, wherein the augmented media content further comprises the at least one instruction.

3. The method of claim 1, wherein the plurality of parameters comprises a plurality of frequencies of the sound wave, wherein the generating of the sound wave comprises:
generating a first sound wave based on the first control signal, wherein the first sound wave is characterized by a first frequency; and
generating a second sound wave based on the first control signal, wherein the second sound wave is characterized by a second frequency, wherein the first frequency is different from the second frequency, wherein the viewer receives the first sound wave through a first ear and the second sound wave through a second ear, wherein the enhancing of the health conditions of the viewer is based on the receiving of the first sound wave through the first ear and the second sound wave through the second ear.

4. The method of claim 1 further comprising:
receiving, using the communication device, at least one viewer data associated with the viewer from the viewer device;
analyzing, using the processing device, the at least one viewer data using at least one second machine learning model; and
determining, using the processing device, at least one requirement of the viewer for the enhancing of the health conditions of the viewer and the at least one viewer context variable corresponding to the at least one requirement based on the analyzing of the at least one viewer data, wherein the generating of the augmented media content is further based on the determining of the at least one requirement of the viewer, wherein the generating of the augmented media content comprises generating the plurality of parameters of the plurality of augmented media content based on the at least one requirement of the viewer.

5. The method of claim 1, wherein the generating of the augmented media content comprises segmenting the media content into a plurality of content phrases, wherein the at least one viewer sensor is further configured for measuring at least one viewer state value during the presenting of each of the plurality of content phrases, wherein the at least one viewer context variable comprises the at least one viewer state value, wherein the method further comprises:
computing, using the processing device, a phrase-state correlation between the at least one viewer state value and each of the plurality of content phrases;
storing, using the storage device, the phrase-state correlation in a phrase-attribution database; and
selecting, using the processing device, a subsequent augmented media content based on the phrase-state correlation between the at least one viewer state value and each of the plurality of content phrases.

6. The method of claim 1 further comprising:
generating, using the processing device, a cryptographic audit record for the augmented media content, wherein the cryptographic audit record comprises at least one of an input data hash representing the at least one viewer context value, a reasoning chain capturing intermediate computational states of the at least one first machine learning model, an output recommendation hash, a cryptographic signature, and a distributed ledger transaction identifier; and
storing, using the storage device, the cryptographic audit record in a distributed ledger.

7. The method of claim 1, wherein the response of the viewer corresponding to the augmented media content comprises a response of the viewer to at least one of the sound wave, the light, and the vibration, wherein the detecting of the response comprises detecting a change in at least one of a physical state, a psychological state, and a biological state of the viewer in response to at least one of the sound wave, the light, and the vibration, wherein the generating of the at least one viewer context value for the at least one viewer context variable is further based on the detecting of the change in at least one of the physical state, the psychological state, and the biological state of the viewer in response to at least one of the sound wave, the light, and the vibration.

8. A system for providing content to viewers for enhancing health conditions of the viewers, the system comprising:
a communication device configured for:
receiving a media content from a broadcaster device;
receiving at least one broadcaster preference from the broadcaster device, wherein the at least one broadcaster preference comprises at least one viewer context variable;
transmitting an augmented media content to a viewer device associated with a viewer, wherein the viewer device is configured for presenting the augmented media content to the viewer, wherein the presenting of the augmented media content on the viewer device is based on at least one viewer context value corresponding to the at least one viewer context variable, wherein the at least one viewer context variable corresponds to at least one viewer sensor comprised in the viewer device, wherein the at least one viewer sensor is configured for generating the at least one viewer context value for the at least one viewer context variable based on detecting a response of the viewer corresponding to the augmented media content, wherein the viewer device is configured for generating a plurality of control signals based on the augmented media content, wherein the plurality of control signals comprises a first control signal, a second control signal, and a third control signal, wherein the viewer device further comprises:
an audio output device configured for generating a sound wave based on the first control signal to drive a first sensory stimulus in the viewer;
a visual output device configured for generating a light based on the second control signal to drive a second sensory stimulus in the viewer; and
a vibrotactile device configured for generating a vibration based on the third control signal to drive a third sensory stimulus in the viewer, wherein the plurality of sensory stimuli comprises the first sensory stimulus, the second sensory stimulus, and the third sensory stimulus, wherein a plurality of sensory stimuli enhances the health conditions of the viewer;
transmitting the augmented media content to the broadcaster device; and
receiving the at least one viewer context value corresponding to the at least one viewer context variable from the viewer device;
a processing device communicatively coupled with the communication device, wherein the processing device is configured for:
generating the augmented media content for the enhancing of the health conditions of a viewer based on the at least one viewer context variable and the media content, wherein the augmented media content comprises the media content and the at least one viewer context variable, wherein the augmented media content further comprises a plurality of parameters corresponding to a plurality of augmented media content, wherein the plurality of parameters of the plurality of augmented media content is configured to drive the plurality of sensory stimuli in the viewer;
analyzing the at least one viewer context value and the at least one viewer context variable corresponding to the at least one viewer context value using at least one first machine learning model;
generating a health data for the viewer based on the analyzing of the at least one viewer context value, wherein the health data includes a health profile of the viewer;
analyzing a plurality of health data; and
generating a plurality of augmentation content based on the analyzing of the plurality of health data; and
a storage device communicatively coupled with the processing device, wherein the storage device is configured for:
storing the health data of the viewer in a distributed ledger;
retrieving the plurality of health data of a plurality of viewers; and
storing the plurality of augmentation content in at least one database.

9. The system of claim 8, wherein the generating of the augmented media content comprises:
generating a plurality of programmable parameters corresponding to the plurality of augmented media based on the at least one viewer context variable and the media content, wherein the plurality of parameters comprises the plurality of programmable parameters; and
generating at least one instruction based on the plurality of programmable parameters, wherein the at least one instruction is configured to synchronize the vibration with the sound wave and the light, wherein the augmented media content further comprises the at least one instruction.

10. The system of claim 8, wherein the plurality of parameters comprises a plurality of frequencies of the sound wave, wherein the generating of the sound wave comprises:
generating a first sound wave based on the first control signal, wherein the first sound wave is characterized by a first frequency; and
generating a second sound wave based on the first control signal, wherein the second sound wave is characterized by a second frequency, wherein the first frequency is different from the second frequency, wherein the viewer receives the first sound wave through a first ear and the second sound wave through a second ear, wherein the enhancing of the of the health conditions of the viewer is based on the receiving of the first sound wave through the first ear and the second sound wave through the second ear.

11. The system of claim 8, wherein the communication device is configured for receiving at least one viewer data associated with the viewer from the viewer device, wherein the processing device is further configured for:
analyzing the at least one viewer data using at least one second machine learning model; and
determining at least one requirement of the viewer for the enhancing of the health conditions of the viewer and the at least one viewer context variable corresponding to the at least one requirement based on the analyzing of the at least one viewer data, wherein the generating of the augmented media content is further based on the determining of the at least one requirement of the viewer, wherein the generating of the augmented media content comprises generating the plurality of parameters of the plurality of augmented media content based on the at least one requirement of the viewer.

12. The system of claim 8, wherein the processing device is further configured for generating a plurality of predefined parameters corresponding to a plurality of predetermined media content based on the analyzing of the plurality of health data, wherein the plurality of predefined parameters is configured to enhance a plurality of health conditions, wherein the storage device is further configured for storing the plurality of predefined parameters in the at least one database.

13. The system of claim 8, wherein the processing device comprises a cryptographic verification module, wherein the cryptographic verification module is configured for:
retrieving a cryptographic audit record from a distributed ledger;
verifying a cryptographic signature based on the retrieving of the cryptographic audit record, wherein the cryptographic audit record comprises the cryptographic signature;
reconstructing a reasoning chain based on the verifying, wherein the reasoning chain indicates intermediate computational states of the at least one first machine learning model; and
generating a verification report based on the reconstruction of the reasoning chain and the verifying of the cryptographic audit record, wherein the verification report confirms an accordance of the augmented media content with the reasoning chain.

14. The system of claim 8, wherein the response of the viewer corresponding to the augmented media content comprises a response of the viewer to at least one of the sound wave, the light, and the vibration, wherein the detecting of the response comprises detecting a change in at least one of a physical state, a psychological state, and a biological state of the viewer in response to at least one of the sound wave, the light, and the vibration, wherein the generating of the at least one viewer context value for the at least one viewer context variable is further based on the detecting of the change in at least one of the physical state, the psychological state, and the biological state of the viewer in response to at least one of the sound wave, the light, and the vibration.

15. A device for providing content to viewers for enhancing health conditions of the viewers, the device comprising:
a communication unit configured for:
receiving at least one media signal from a broadcaster device, wherein the at least one media signal corresponds to a media content; and
receiving at least one broadcaster preference from the broadcaster device, wherein the at least one broadcaster preference comprises at least one viewer context variable;
a processing unit communicatively coupled with the communication unit, wherein the processing unit is configured for:
generating an augmented media signal for the enhancing of the health conditions of a viewer based on the at least one media signal and the at least one broadcaster preference; and
generating a plurality of control signals based on the augmented media signal;
at least one visual output device communicatively coupled with the processing unit, wherein the visual output device is configured for generating a light based on a first control signal, wherein the plurality of control signals comprises the first control signal, wherein the viewer is associated with the at least one visual output device;
at least one audio output device communicatively coupled with the processing unit, wherein the at least one audio output device is configured for generating at least one sound wave based on a second control signal, wherein the plurality of control signals further comprises the second control signal, wherein the viewer is further associated with the at least one audio output device;
at least one vibrotactile device communicatively coupled with the processing unit, wherein the at least one vibrotactile device is configured for generating at least one vibration based on a third control signal, wherein the plurality of control signals further comprises the third control signal, wherein the viewer is further associated with the at least one vibrotactile device, wherein the light, the at least one sound wave, and the at least one vibration enhances the health conditions of the viewer; and
at least one sensor device communicatively coupled with the processing unit, wherein the at least one sensor device is configured for:
detecting at least one response of the viewer to at least one of the light, the at least one sound wave, and the at least one vibration; and
generating at least one viewer context value for the at least one viewer context variable based on the detecting of the at least one response, wherein the generating of the augmented media signal for the enhancing of the health conditions is further based on the at least one viewer context value.

16. The device of claim 15, wherein the at least one vibrotactile device comprises a bed configured to vibrate based on the third control signal, wherein the bed comprises at least one of water and a foam.

17. The device of claim 15, wherein the detecting of the response comprises detecting a change in at least one of a physical state, a psychological state, and a biological state of the viewer in response to at least one of the sound wave, the light, and the vibration, wherein the wherein the generating of the at least one viewer context value for the at least one viewer context variable is further based on the detecting of the change in at least one of the physical state, the psychological state, and the biological state of the viewer in response to at least one of the sound wave, the light, and the vibration.

18. The device of claim 15, wherein the at least one sensor device comprises a visible-spectrum camera configured for performing facial expression analysis during an eyes-opens state of the viewer, wherein the at least one sensor device further comprises a near-infrared camera for performing physiological monitoring during an eyes-closed state of the viewer, wherein the at least one sensor device further comprises a mode controller configured for switching between the performing of the facial expression analysis and the performing of the physiological monitoring based on an eye state of the viewer, wherein the mode controller ensures continuous biometric monitoring regardless of the eye state of the viewer.

19. The device of claim 15, wherein the generating of the augmented media signal comprises generating a plurality of parameters corresponding to the light, the at least one sound wave, and the at least one vibration based on the at least one media signal and the at least one broadcaster preference, wherein the generating of the plurality of control signals is based on the plurality of parameters.

20. The device of claim 15, wherein the at least one sound wave is configured to drive a first sensory stimulus in the viewer, wherein the light is configured to drive a second sensory stimulus in the viewer, wherein the at least one vibration is configured to drive a third sensory stimulus in the viewer, wherein the first sensory stimulus, the second sensor stimulus, and the third sensory stimulus enhances the health conditions of the viewer.
